(19) United States
(12) Patent Application Publication
(10) Pub. No.: US 2025/0269056 A1
(43) Pub. Date: Aug. 28, 2025

(54) NUCLEIC ACID DELIVERY SYSTEM

(71) Applicants: Roderick SLAVCEV, Kitchener (CA); Shirley WONG, Kitchener (CA)

(72) Inventors: Roderick SLAVCEV, Kitchener (CA); Shirley WONG, Kitchener (CA)

(21) Appl. No.: 18/859,873

(22) PCT Filed: May 1, 2023
(86) PCT No.: PCT/CA2023/050586

§ 371 Date: Oct. 24, 2024

Related U.S. Application Data
(60) Provisional application No. 63/336,844, filed on Apr. 29, 2022.

(51) Int. Cl. A61K 48/00; C12N 15/113; C12N 15/70; C12N 15/86

(52) U.S. Cl. CPC A61K 48/0041; C12N 15/113; C12N 15/70; C12N 15/86; C12N 2310/11; C12N 2795/14143; C12N 2795/14145; C12N 2795/14152; C12N 2800/202

(57) ABSTRACT

Disclosed herein is a minivector for use in delivering a deoxyribonucleic acid to at least one target cell, the minivector comprising the deoxyribonucleic acid and a filamentous phage origin of replication, wherein the minivector is otherwise devoid of prokaryotic vector backbone genetic sequences. The minivector may be packaged or encapsulated in a filamentous bacteriophage. Also disclosed are minivector precrusors, filamentous bacteriophage particles encapsulating minivectors, methods of producing minivectors and bacteriophage comprising minivectors, methods of delivering a deoxyribonucleic acid to a mammalian cell, methods treating or preventing a disease or condition. Compositions, kits and commercial packages and are also disclosed.

### CROSS REFERENCE TO RELATED APPLICATIONS

All publications, patents, and patent applications mentioned in this specification and exhibits are herein incorporated by reference to the same extent as if each individual publication, patent, or patent application is specifically and individually indicated to be incorporated by reference.

### FIELD

The present disclosure relates generally to the delivery of deoxyribonucleic acids to cells. More particularly, the present disclosure relates to targeted delivery of deoxyribonucleic acids to mammalian cells.

### BACKGROUND

The delivery of deoxyribonucleic acids to mammalian cells is important for applications including, for example, gene therapy, gene editing including gene knock-ins, treatment or prevention of disease, vaccination, drug development, diagnosis of disease and monitoring of disease progression.

Physical methods which force exogenous DNA into the cytoplasm of cells are known, including direct DNA injection, jet-injection, or electroporation. Chemical methods of delivery rely on encapsulation of genetic material to assist its endocytic uptake and endosomal escape. However, physical and chemical methods generally have a low efficiency of delivery and tend to be restricted in use due to tissue accessibility and the capacity for tissue damage.

Delivery vehicles such as viral vectors derived from retroviruses, adenoviruses, vaccinia viruses, herpes viruses, and adeno-associated viruses have been described. However, viral vectors exhibit safety concerns such as high immunogenicity, risk of insertional mutagenesis, and the possibility of recombination into replication competent viruses. Viral vectors also exhibit limitations with respect to cargo capacity which together with the immunogenicity limit their efficacy.

Non-viral DNA delivery uses conventional plasmids having a bacterial backbone are not as immunogenic as viral vectors. However, the efficiency of gene expression using plasmid delivery is low and there is a risk of insertional mutagenesis due to its closed circular nature.

A need exists for nucleic acid delivery systems and methods for delivering deoxyribonucleic acids to cells.

### SUMMARY

Disclosed herein is a minivector. The minivector may be encapsulated by filamentous bacteriophage proteins or packaged in a filamentous bacteriophage and may be used for delivering a deoxyribonucleic acid to at least one target cell. The minivector comprises a deoxyribonucleic acid and a filamentous phage origin of replication and the minivector is otherwise devoid of prokaryotic vector backbone genetic sequences. The at least one target cell may be a mammalian cell, for example, a human cell. The minivector may be produced from a minivector precursor comprising a split origin of replication. The minivector precursor may comprise a deoxyribonucleic acid flanked by initiation and termination elements of a filamentous phage origin of replication.

Disclosed herein is a minivector for use in delivering a deoxyribonucleic acid to at least one target cell, the minivector comprising the deoxyribonucleic acid and a filamentous phage origin of replication, wherein the minivector is otherwise devoid of prokaryotic vector backbone genetic sequences. The minivector may be a single-stranded DNA minivector, a double stranded DNA minivector, a circularly covalently closed single-stranded minivector or a linear covalently closed double stranded DNA minivector. The filamentous phage origin of replication may be an M13 origin of replication. The filamentous phage origin of replication may be an f1 origin of replication. The minivector may be encapsulated by filamentous phage proteins.

Also disclosed is a filamentous bacteriophage particle comprising a minivector. The filamentous bacteriophage particle may comprise a targeting moiety on the bacteriophage particle surface for targeting the bacteriophage particle to at least one target cell. In an embodiment, the targeting moiety is epidermal growth factor and the at least one target cell expresses epidermal growth factor receptor.

Further disclosed is a minivector precursor. In an embodiment, the minivector precursor comprises a polynucleotide comprising X-Y-Z,
wherein X comprises an initiation element of a filamentous phage origin of replication;
Y comprises a cassette comprising a deoxyribonucleic acid; and
Z comprises a termination element of the filamentous phage origin of replication. In other words, the minivector precursor comprises a deoxyribonucleic acid for delivery to a target cell, wherein the deoxyribonucleic acid is flanked by an initiation element of a filamentous phage origin of replication and a termination element of the filamentous phage origin of replication. Helper bacteriophage generate a recombinant molecule from the split origin of replication of the minivector precursor that is assembled into a minivector phage particle. Phage-encapsulated minivector may be used to deliver the deoxyribonucleic acid to a mammalian cell.

Also disclosed is a method of producing a minivector encapsulated in a filamentous bacteriophage for use in delivering a deoxyribonucleic acid to at least one target cell, the method comprising: infecting a bacterial host cell comprising a minivector precursor with a helper bacteriophage, wherein the bacterial host cell is capable of propagating filamentous phage and wherein the minivector precursor comprises a deoxyribonucleic acid flanked by isolated initiation and termination elements of a filamentous phage origin of replication and wherein the helper phage is a filamentous bacteriophage; culturing the bacterial host cell under conditions that promote replication and amplification of the minivector from the minivector precursor, packaging of the minivector into progeny phage and extrusion of the progeny phage from the host cell; and harvesting extruded progeny filamentous bacteriophage encapsulating the minivector.

The filamentous bacteriophage may comprise a targeting moiety on the phage surface for targeting the phage to the at least one target cell. In an embodiment, the helper phage is f1, fd or M13, such as M13KO7. In an embodiment the helper phage lacks a packaging signal. In an embodiment, the helper phage is derived from M13KO7 and has been modified to replace the packaging signal downstream of gIV and upstream of Tn903 with a Rho-independent terminator. In an embodiment, the helper phage is M13SW8 and comprises the sequence set forth in SEQ ID NO:4. In an embodiment, the helper phage comprises a targeting moiety on the bateriophage cell surface for targeting the bacteriophage encapsulating the minivector to at least one target cell. In an embodiment, the targeting moiety is epidermal growth factor and the at least one target cell expresses epidermal growth factor receptor.

In an embodiment, the deoxyribonucleic acid comprises a sense copy and an antisense copy of a target deoxyribonucleic acid. In an embodiment, the sense copy and the antisense copy of the target deoxyribonucleic acid are separated by a linker for stabilizing the minivector precursor during cloning. In an embodiment, the helper phage is maintained under antibiotic selective pressure prior to the step of infecting and/or during the infecting and/or culturing steps. In an embodiment, the antibiotic is kanamycin. In an embodiment, the bacterial host cells are infected for about 16 to about 18 hours. In an embodiment, the helper phage are at a concentration of about 1×10⁸ PFU/mL in the step of infecting.

The disclosed minivectors and recombinant phage comprising the minivectors may be used in a method of delivering a target deoxyribonucleic acid to at least one target cell in a subject in need thereof. Also disclosed are pharmaceutical compositions, kits and commercial packages comprising the minivectors and/or bacteriophage particles encapsulating the minivectors. Encapsulated minivectors may be used in any method that requires delivery of a deoxyribonucleic acid to a target cell, including, but not limited to, methods of treating or preventing disease, methods of diagnosing or monitoring disease, methods of vaccination.

Other aspects and features of the present disclosure will become apparent to those ordinarily skilled in the art upon review of the following description of specific embodiments in conjunction with the accompanying figures.

### BRIEF DESCRIPTION OF THE DRAWINGS

A better understanding of the features and advantages of the present invention will be obtained by reference to the following detailed description that sets forth illustrative embodiments, in which the principles of the invention are utilized. Embodiments of the present disclosure will now be described, by way of example only, with reference to the attached Figures.

FIG. 1 is a schematic showing the secondary structure of the f1 origin of replication on the viral plus strand, including the sequences required for termination and initiation of replication;

FIG. 2 shows the nucleotide sequence of terminator and initiator domains of the f1 origin of replication as identified in Short et al., 1988 and including the overlapping region including the pII nicking site;

FIG. 3 shows a schematic of the lifecycle of a filamentous bacteriophage;

FIG. 4 shows maps of exemplary minivector precursors;

FIG. 5 shows the production of recombinant RFx from minivector precursors for producing single-stranded minivectors;

FIG. 6 shows the isolation of single stranded minivectors from phage lysates; A shows purified DNA quantified on nanodrop; and B shows ssDNA visualized on AGE;

FIG. 7 shows the composition of phage lysates encoding either cmv-gfp or cmv-luc. In the top panel, the concentration of each phage species (minivector, gfp and luc; full vector, gfp and luc and helper phage) in each lysate are presented. In the lower panel, the proportion of target phagemid is shown as a percentage of the total phage population;

FIG. 8 shows the production of minivectors comprising gfp-tet. The arrows designate the helper genome, the plasmid or corresponding full phagemid, the recombinant RF, RFx or minivector;

FIG. 9 shows the composition of phage lysates encoding one of cmv-gfp, cmv-luc and cmv-gfp-tet;

FIG. 10 shows the composition of EGF-displaying phage lysates and the ability of targeted phage to assemble miniphagemid;

FIG. 11 shows the construction of a helper phage without a packaging signal (PS);

FIG. 12 shows the composition of lysates packaged by PS-deficient helper phage;

FIG. 13 shows the production of recombinant RFx by PS-deficient helper phage;

FIG. 14 shows the localization of EGF-displaying phage particles in HeLa cells;

FIG. 15 shows gene expression in mammalian cells of phage delivered minivectors comprising transgenes;

FIG. 16 shows gene expression of precursor full vectors and purified minivector DNA over time;

FIG. 17 shows a map of an exemplary SAS minivector precursor;

FIG. 18 shows the production of double-stranded minivectors from a sense-antisense precursor vector and rescue by helper phage;

FIG. 19 shows the packaging of a double-stranded minivector by helper phage;

FIG. 20 shows AGE of phagemid and plasmid constructs following treatment with mung bean nuclease;

FIG. 21 shows the effect of optical density and helper antibiotic on SAS RFx conversion;

FIG. 22 shows the effect of phagemid and helper antibiotic on SAS RFx conversion;

FIG. 23 shows the effect of antibiotic on SAS RFx conversion;

FIG. 24 shows the effect of antibiotic, optical density, and MOI on SAS RFx conversion;

FIG. 25 shows separation of the LCC dsDNA minivector from ssDNA M13KO7 genome using hydroxyapatite chromatography;

FIG. 26 shows exemplary production and analysis of SAS phagemid from M13W8 helper bacteriophage; and

FIG. 27 shows agarose gel electrophoresis of M13SW8 SAS minivectors and miniphagemids.

### DETAILED DESCRIPTION

Generally, the present disclosure provides a deoxyribonucleic acid (DNA) delivery system comprising a miniaturized vector (minivector) for delivery of DNA to a target cell. The minivector may be encapsulated in bacteriophage proteins forming a bacteriophage particle. The bacteriophage proteins encapsulate and protect the nucleic acid cargo encoded by the minivector allowing the delivery to a target cell. In some embodiments the bacteriophage particle may comprise a targeting moiety on the phage surface for specifically targeting the DNA to a target cell.

The minivector comprises a phage origin of replication, but lacks prokaryotic vector backbone genetic sequences found in conventional vectors. Prokaryotic vector backbone genetic sequences include, but are not limited to, genetic elements that are present in conventional plasmids and required for amplification and maintenance of the plasmid in a bacterial host. Prokaryotic vector backbone sequences are rich in unmethylated cytosine-guanine dinucleotide (CpG) motifs that are known to inhibit transgene expression in mammalian cells due in part to the inflammatory response they induce in cells. Minivectors which lack prokaryotic backbone sequences lack the unmethylated CpG motifs found in bacterial sequences, and therefore escape endosomal degradation and avoid CpG-mediated gene silencing.

Prokaryotic vector backbone sequences also contain antibiotic resistance markers that can disseminate antibiotic resistance to the environment. As the disclosed minivectors lack bacterial antibiotic markers, they do not promote antibiotic resistance.

The DNA that is to be delivered to a target cell is flanked between the initiation and termination regions of a phage origin of replication in a precursor minivector. Thus, the minivector comprises a “split origin of replication”. Separation of the initiation and termination signals of the origin of replication results in the replication of only the DNA between these replication signals and results in minivectors which lack a prokaryotic backbone. The disclosed minivectors exhibit improved DNA transfer efficiency compared to vectors with intact wild-type origins of replication and comprising prokaryotic backbones.

Filamentous bacteriophage length is determined largely by the length of the encapsulated DNA molecule. As the disclosed minivectors lack any prokaryotic backbone, the encapsulated DNA is much smaller than that of an intact full vector. The small size of the encapsulated minivectors provides advantages such as conferring improved cytoplasmic diffusion, promoting more efficient internalization upon binding the target cell and allowing for packaging and delivery of larger deoxyribonucleic acids.

A minivector encapsulated by filamentous phage particles may be produced by introducing a minivector precursor into a bacterial host cell capable of propagating filamentous bacteriophage; infecting the bacterial host cell with a filamentous helper bacteriophage; and culturing the bacterial host cell under conditions that promote replication and amplification of the minivector from the minivector precursor, packaging of the minivector into progeny phage and extrusion of the progeny bacteriophage from the bacterial host cell. Extruded progeny bacteriophage comprising the minivector may be harvested from the bacterial host cell. In some embodiments, the minivector may be isolated from the extruded progeny bacteriophage.

The minivector precursor comprises a split origin of replication. A split origin of replication refers to a phage origin of replication wherein an initiation element is physically separated from a termination element of a phage origin of replication. Separation of the initiation and termination elements to form the split origin of replication enables the replication of only DNA found between the two replication signals, producing smaller minivectors. The deoxyribonucleotide for delivery is between the separated fragments of the initiation and termination elements of the phage origin of replication. In other words the deoxyribonucleic acid is flanked by the initiation and termination elements of the origin of replication.

In an embodiment, the split origin of replication is a split filamentous phage origin of replication. In an embodiment the split origin of replication is a split M13 origin of replication. In an embodiment, the split origin of replication is a split f1 functional origin of replication (also known as the f1 ori, or the f1 origin of replication). The minivector precursor comprises a deoxyribonucleic acid for delivery flanked by initiation and termination elements of a filamentous phage origin of replication. In some embodiments the minivector precursor comprises a cassette comprising a deoxyribonucleic acid for delivery. In some embodiments the deoxyribonucleic acid for delivery comprises a target deoxyribonucleic acid which specifies a deoxyribonucleic acid sequence to be expressed in a target cell. The deoxyribonucleic acid may comprise genetic elements required for expression of a target deoxyribonucleotide in a target cell once it is delivered.

In an embodiment, the precursor minivector comprises a polynucleotide comprising X-Y-Z, wherein
X comprises an initiation element of a filamentous phage origin of replication;
Y comprises a cassette comprising a deoxyribonucleic acid; and
Z comprises a termination element of the filamentous phage origin of replication.

The split origin of replication directs phage proteins to replicate only the deoxyribonucleotides present between the initiation element and the termination element. The backbone of the vector is not replicated. Thus, flanking a cassette with separated initiation and termination regions enables production of a minivector without any vector backbone and results in high titres of minivectors for use as DNA delivery vectors.

The deoxyribonucleic acid comprises a deoxyribonucleic acid that is to be delivered to the cell. Examples of deoxyribonucleic acids that may be delivered include DNA molecules that may be used for therapeutic or diagnostic purposes or may be used as a vaccine. The deoxyribonucleic acid or the cassette may comprise genetic regulatory elements, including, for example a promoter or a polyadenylation signal. In an embodiment the cassette comprises a promoter for expressing the target deoxyribonucleotide in a target cell. An example of a promoter is the cmv promoter, however any suitable promoter may be used. In an embodiment, the cassette comprises a polyadenylation signal, for example, an SV40 polyadenylation signal. Similarly to the phage origin of replication, the split origin of replication allows replication by filamentous phage machinery independently of its plasmid origin. The filamentous phage machinery comprises replication carried out by filamentous phage proteins pII and pX.

In an embodiment, the minivector may be a sense-antisense (SAS) minivector precursor and may comprise sense-antisense (SAS) sequences of a deoxyribonucleic acid. A sense-antisense precursor minivector comprises a split origin of replication. The sense and antisense sequences of the deoxyribonucleic acid are flanked by the initiation and termination domains of a filamentous bacteriophage origin of replication. The complementary sequences of the sense and antisense nucleotide sequences bind and form a linear covalently-closed double stranded DNA minivector. In an embodiment, sense anti-sense circular ssDNA will form a mostly linear covalently closed (LCC) DNA minivector that can be purified from the host cell as a scalable LCC minivector prior to packaging by capsid proteins into a functional phagemid. In an embodiment, LCC DNA minivectors may be used as vectors for rAAV production, or mRNA production.

In some embodiments, a sense-antisense precursor minivector comprises a linker between sense and antisense (SAS) sequences of a target deoxyribonucleic acid. A “linker” is a deoxyribonucleotide sequence that provides stability to the SAS construct by interrupting the palindromic sequence. The linker may be non-palindromic. The linker may be between 50 bp and 300 bp in length. In an embodiment, the linker is between 50 bp and 264 bp in length. In an embodiment, the linker is 50 bp in length. In an embodiment the linker is 264 bp in length.

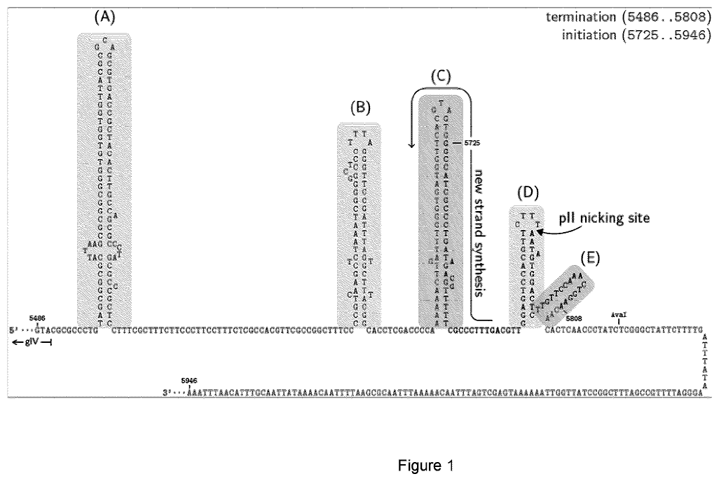

FIG. 1 shows the secondary structure of the f1 functional origin of replication on the viral plus strand. Hairpin structures are formed by palindromic sequences, including (from 5′ to 3′ beginning immediately downstream of gIV: (A) packaging signal, (B) binding site for RNA polymerase, (C) binding site of RNA primer synthesis, (D) location of pII nicking site, (E) binding site for pII. As indicated bythe arrow in FIG. 1, pII nicks at a site within the overlap of the termination and initiation domains to initiate new strand synthesis, which proceeds as indicated by the hook arrow. The sequence and secondary structure domains are described in Dotto et al., 1984; Horiuchi K., 1997 and Short and Sorge, 1995; Beck and Zinck, 1981. The domain containing cis-acting elements to initiate replication begins with hairpin loops C-E that comprise the site of action for phage replication protein, plI.

An initiation element comprises the minimal region of the origin of replication required for the initiation of replication. A termination element comprises the minimal stop region within the origin of replication required to terminate replication. The split origin of replication separates the sequences known only to participate in replication initiation from those known only to participate in termination thereof. Of the hairpin loops found in the f1 ori, only the loops near the pII nicking site, as well as the domain downstream, are necessary for the initiation of replication. Only the D loop containing the pII nicking site and the domain immediately upstream are necessary for the termination of replication.

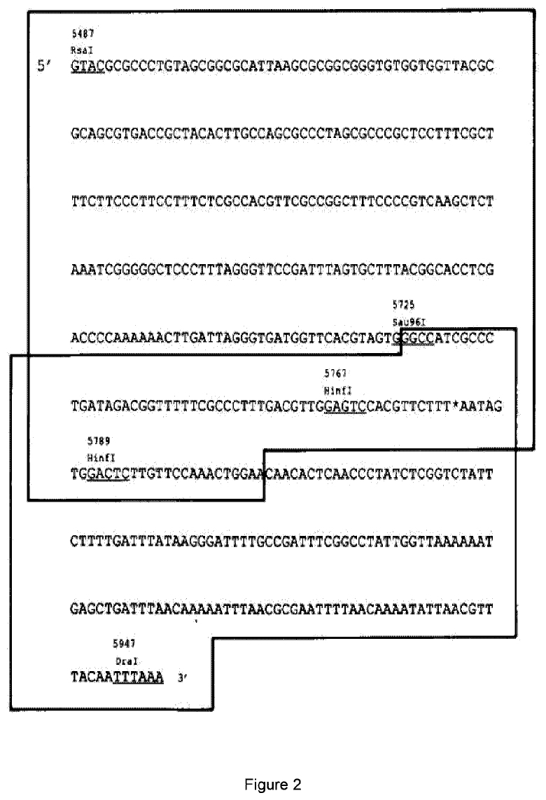

FIG. 2 shows the sequence of the terminator and initiator domains of the f1 origin as identified by Short et al., 1988. In FIG. 2, the nicking site is indicated (*) and is found in the overlapping region. In an embodiment, the termination element comprises nucleotides 5486 to 5808 of the M13 f1 origin of replication. In an embodiment, the initiation element comprises nucleotides 5725 to 5946 of the f1 origin of replication.

In an embodiment, the minivector is encapsulated by filamentous bacteriophage proteins. A minivector encapsulated in bacteriophage proteins may be produced by introducing a minivector precursor into a bacterial host cell capable of propagating filamentous bacteriophage; infecting the bacterial host cell with a filamentous helper bacteriophage; and culturing the bacterial host cell under conditions that promote replication and amplification of the minivector from the minivector precursor, packaging of the minivector into progeny phage and extrusion of the progeny phage from the host cell. Extruded progeny bacteriophage comprising the minivector may then be harvested. The minivector may be isolated from the extruded progeny bacteriophage.

In an embodiment, there is provided a method of producing a minivector encapsulated in a filamentous bacteriophage particle, the method comprising:
infecting a bacterial host cell comprising a minivector precursor with a helper phage, wherein the bacterial host cell is capable of propagating filamentous bacteriophage and wherein the minivector precursor comprises a deoxyribonucleic acid flanked by initiation and termination elements of a filamentous bacteriophage origin of replication and wherein the helper phage is a filamentous bacteriophage;
culturing the bacterial host cell under conditions that promote replication and amplification of the minivector from the minivector precursor, packaging of the minivector into progeny bacteriophage and extrusion of the progeny bacteriophage from the bacterial host cell; and
harvesting extruded progeny filamentous bacteriophage encapsulating the minivector.

A minivector is a miniaturized vector and is used interchangeably with “phagemid minivector”. The minivector is a DNA minivector and, thus may also be defined as a “phagemid DNA minivector” or “DNA minivector”. Minivectors may be single-stranded or double-stranded. Minivectors may be double-stranded covalently closed (CCC) or linear covalently closed (LCC) molecules. A “miniphagemid” is a minivector encapsulated with phage capsid proteins.

A bacteriophage is a virus that specifically infects bacteria. “Ff” or “filamentous bacteriophages” (Inoviridae) are long non-lytic viruses that primarily infect Gram-negative bacteria. Examples of filamentous bacteriophages include f1, fd, M13 and ZJ/2. The terms “bacteriophage” and “phage” are used interchangeably herein.

A “targeted bacteriophage” is a bacteriophage that comprises a cell-targeting moiety present on the bacteriophage surface. A “cell-targeting moiety” is a moiety that specifically targets the phage to at least one target cell. A cell-targeting moiety may permit the specific interaction of a targeted bacteriophage with a target cell and allows the delivery of the deoxyribonucleic acid to the specific target cell. This allows for targeted, delivery of deoxyribonucleic acids encapsulated with bacteriophage proteins that comprise a cell-target moiety. In an embodiment, the cell-targeting moiety is a ligand, and accordingly, the target cell would express a receptor for the ligand on the cell surface. The binding of the ligand and receptor is specific and allows for the delivery of the deoxyribonucleic acid to a specific target cell. The cell-targeting moiety may be, for example, a cell-specific ligand. A cell-specific ligand may take advantage of clathrin-coated receptor mediated endocytosis for cell entry. In an embodiment, the helper phage displays a cell-specific targeting ligand. In an embodiment the cell-specific targeting ligand is epidermal growth factor (EGF). In an embodiment, the cell-specific targeting ligand targets the bacteriophage to a target cell which expresses the EGF receptor on its cell surface. This allows specific targeting of the deoxyribonucleic acid encapsulated with phage proteins comprising EGF to the EGFR-expressing target cell. In an embodiment, the cell-targeting moiety may be an antibody or antibody fragment and the target cell expresses a molecule that specifically interacts with the antibody or antibody fragment. The bacteriophage may be engineered such that the cell targeting moiety is part of the phage surface using, for example, phage display, or any other suitable technique. For example, the cell targeting moiety may be covalently bound to the phage surface. The cell-targeting moiety may promote phage:cell interactions. For example, the cell-targeting moiety may be sialic acid.

A “target cell” is a cell to which the deoxyribonucleic acid is to be delivered. In some embodiments, the target cell expresses a target molecule on its cell surface for targeted binding of the phage particle to the target cell and promoting targeted delivery of the deoxyribonucleic acid to the target cell. A target molecule is a molecule to which the targeting moiety binds. Such molecules may be cell surface molecules, such as, e.g., polypeptides, lipids, or polysaccharides that can be specifically bound by the targeting moiety. In an embodiment, the target cell is a cell which expresses epidermal growth factor receptor (EGFR) on its cell surface.

The deoxyribonucleic acid may comprise any regulatory elements required for the expression of the deoxyribonucleic acid to be delivered in a target cell, thereby forming an expression cassette. The regulatory elements may comprise a promoter, enhancers, a 5′ UTR, 3′ UTR, and/or additional genetic elements such as: scaffolding/matrix attachment regions, chromatin opening elements, post-transcriptional regulatory elements and/or a polyadenylation sequence.

A plasmid is a circular covalently closed double-stranded DNA molecule typically amplified in bacterial cells. A phagemid is a plasmid also comprising a phage origin of replication and may be encapsulated by phage proteins. A phage origin of replication allows replication by filamentous phage machinery independent of its plasmid origin.

A helper phage is a bacteriophage that provides the phage proteins in trans that are necessary for amplification of minivectors. The helper phage encodes all phage proteins necessary for f1 ori-mediated replication and phage assembly and extrusion. In an embodiment, the helper phage is a filamentous bacteriophage. The helper phage may be modified, for example to be interference-resistance or to increase replication efficiencies, for example by disruption of the IR. In an embodiment the helper phage is M13KO7. M13KO7 is a derivative of phage M13 with the origin of replication from p15a and a kanamycin gene inserted in the Ava1 site (5825) of the IR. In an embodiment the helper phage is M13KE. M13KE contains an interrupted IR and this leads to deficient self replication in the presence of a wild-type uninterrupted f1 origin. In an embodiment the helper phage is M13SW8. Other examples of helper phages include display-specific helper phages such as R408d3 and CT.

A bacterial host strain is a strain that is capable of propagating filamentous Ff bacteriophages. The bacterial host must be susceptible to Ff phage infection. F-specific filamentous phages (Ff phages) have the capacity to infect any Gram-negative bacteria that expresses cell-surface ToIA. For example, the host strain may be an E. coli host strain or a Salmonella typhimurium host strain.

The E. coli host strain may be derived from E. coli K12. Examples of E. coli host strains include, but are not limited to JM109, JM110, XL1-Blue, Stbl2, Stbl3, Stbl4, NEB Turbo, ER2738, TG1, NM522, CSH50, TG1, HB101, MG1655 and DH5a. In an embodiment, the E. coli host strain is an F+E. coli. In an embodiment, the E. coli host strain is a recombination deficient strain (recA). In an embodiment, the E. coli host strain is a recA, gyrA, endA host strain. Mutations such as recA and gyrA reduce occurrences of homology-dependent recombination events. In an embodiment the E. coli host strain is JM109. In an embodiment, the E. coli host strain is JM109 and the helper phage is M13SW8. In an embodiment, the E. coli host strain is an sbCD or recF host strain, such as SURE or SURE2.

JM109 and XL1-Blue are recombination-deficient (recA) strains and have similar published genotypes. Both carry the gyrA96 mutation, where gyrA encodes for the GyrA subunit of DNA gyrase. DNA gyrase has been implicated in homology-dependent, RecA-independent deletions; the gyrA96 mutation gives resistance to nalidixic acid and stabilizes the cloning of repetitive DNA although does not adversely impact supercoiling of plasmid DNA. NM522 and TG1 are both recombination-competent strains often used for the propagation of filamentous phage. NM522 is hsdA5 derivative of 71-18, a (lac-pro) strain of E. coli, commonly used for the production of single-stranded DNA and is commercially available from a number of companies. HB101 is a cross between E. coli K12 and B. Stbl3 is a recA derivative of HB101 with similar properties to Stbl4. Mach1 is a fast-growing strain similar to NEB Turbo, which is derived from E. coli W.

ER2738 is a derivative of NM522 with a TcR marker present on its F′ episome and commercially sold as a host for M13 phage display. The NEB Turbo strain from New England BioLabs is notable for its rapid growth rate and good plasmid yield; its genotype bears resemblance to TG1, which was also recognized for its rapid growth. Stbl4 is derived from Stbl2, which itself is a derivative of JM109; both strains were developed for applications with large repeats of DNA or large plasmids that are normally unstable in E. coli strains.

A packaging signal (PS) is a nucleotide sequence in a helper phage that is recognized by phage assembly proteins. Deletion of the packaging signal in the helper phage prevents active extrusion of helper phage out of the cell reducing or eliminating helper phage contamination of the resultant lysate, thereby leading to an improved phagemid:helper ratio.

Disclosed herein is a method of producing a minivector packaged in a filamentous phage for use in delivering a deoxyribonucleic acid to at least one target cell, the method comprising: infecting a bacterial host cell comprising a minivector precursor with a helper phage, wherein the E coli host cell is capable of propagating filamentous phage and wherein the minivector precursor comprises a cassette flanked by isolated initiation and termination elements of a Ff phage origin of replication and wherein the isolated initiation and termination domains each comprise a pII nicking site and wherein the helper phage is a filamentous bacteriophage;
culturing the bacterial host cell under conditions that promote replication and amplification of the minivector from the minivector precursor, packaging of the minivector into progeny phage and extrusion of the progeny phage from the host cell; and
harvesting extruded progeny filamentous phage comprising the minivector.

Disclosed herein is a method of producing a minivector for use in delivering a deoxyribonucleic acid to at least one target cell, the method comprising:
infecting a bacterial host cell comprising a minivector precursor with a helper phage, wherein the bacterial host cell is capable of propagating filamentous phage, wherein the minivector precursor comprises a cassette flanked by isolated initiation and termination elements of a Ff phage origin of replication and wherein the isolated initiation and termination domains each comprise a pII nicking site and wherein the helper phage is a filamentous bacteriophage;
culturing the bacterial host cell under conditions that promote replication and amplification of the minivector from the minivector precursor, packaging of the minivector into progeny phage and extrusion of the progeny phage from the host cell;
harvesting extruded progeny phage;
separating bacterial debris from phage lysate by centrifugation to produce a supernatant containing the phage lysate;
optionally purifying the supernatant containing the phage lysate by filtration to obtain a filtered lysate;
optionally concentrating the filtered lysate; and
isolating the minivector from the phage lysate.

In an embodiment, the method comprises transforming a bacterial host cell with a minivector precursor encoding a deoxyribonucleic acid between separated initiation and termination elements (for example, M13 START and STOP domains). After infection with a helper phage, phage protein expression can occur. Rolling circle amplification generates a recombinant replicative factor (RFx) from the precursor minivector that reconstitutes the origin of replication and the plasmid backbone is lost. The phage ssDNA binding protein pV sequesters single-stranded minivector preventing further amplification. Assembly proteins extrude progeny phage particles encapsulating the minivector. In an embodiment, the helper phage is maintained under antibiotic selective pressure prior to the step of infecting and/or during the infecting and/or culturing steps. In an embodiment, the antibiotic is kanamycin.

Disclosed herein are filamentous bacteriophages that encapsulate minivectors for delivery of DNA to mammalian cells. The filamentous phages serve as gene delivery vehicles wherein the phage particles form a proteinaceous coat surrounding a nucleic acid core. Phage particles can contain single stranded DNA or double stranded DNA. Bacteriophages do not have any tropism for eukaryotic cells and are not able to replicate in eukaryotic cells. In addition, phages are generally inexpensive to manufacture in large quantities as they replicate in bacterial culture which is highly amenable to scale up. Phages exhibit good stability in the face of environmental factors such as pH and temperature. Bacteriophages are also amenable to dessication allowing inexpensive, long-term storage.

Filamentous bacteriophages include F-specific filamentous phages (Ff phages). Filamentous phages do not lyse their hosts; after infection, progeny continuously bud out from the infected host over the course of the infected bacterium's lifetime. Examples of filamentous phages that specifically infect E. coli include f1, fd, and M13. F1, fd and M13 share 98% sequence homology and have been studied interchangeably. Viruses such as the filamentous bacteriophage M13 exist as inert virions outside their host cell, E. coli. In order to propagate, filamentous bacteriophage infect a bacterial host, replicate within the cell, and finally assemble phage progeny which can then extrude from the cell. The M13 genome encodes all eleven proteins necessary for these activities, which are controlled by signalling structures within a short non-coding intergenic region (IR).

Filamentous bacteriophages comprise approximately 2700 copies of the major coat protein pVIII (50 amino acids) which wrap around the ssDNA core to form the tubular coat protein. Each pVIII unit is comprised of a hydrophilic N-terminal domain oriented externally to the virion particle and a positively charged C-terminus facing inwards in the virion particle to interact with packaged phage DNA. Filamentous bacteriophages generate high titres of phage progeny. Filamentous bacteriophage capsid proteins are amenable to the display of foreign peptides to modify tropism. Filamentous bacteriophages also comprise a simple, well-characterized genome that is easy to genetically modify. Filamentous bacteriophage (e.g. M13) virion size is flexible and is entirely dictated by genome length, enabling a theoretically limitless capacity for DNA cargo.

M13 bacteriophage is comprised of a circular closed single-stranded genome (6407 nucleotides) encoding 11 gene products, 5 of which are capsid proteins. The genes are numbered I-XI and denoted by the prefix “g” (ex. gIII), while their products are denoted by the prefix “p” (ex. pIII). Proteins pII and pX are involved in the amplification of the replicative factor (RF), the circular DNA episomal form of the phage genome. Replicative protein pII (410 a.a.) is necessary and sufficient for rolling circle amplification of the filamentous phage genome. pX also regulates replication. pV sequester amplified single-stranded viral genome molecules for phage assembly. Proteins pI, pXI and pIV mediate assembly and export of progeny phage, along with the capsid proteins) pIII, pVI, pVII, pVIII, and pIX) filamentous bacteriophage genome.

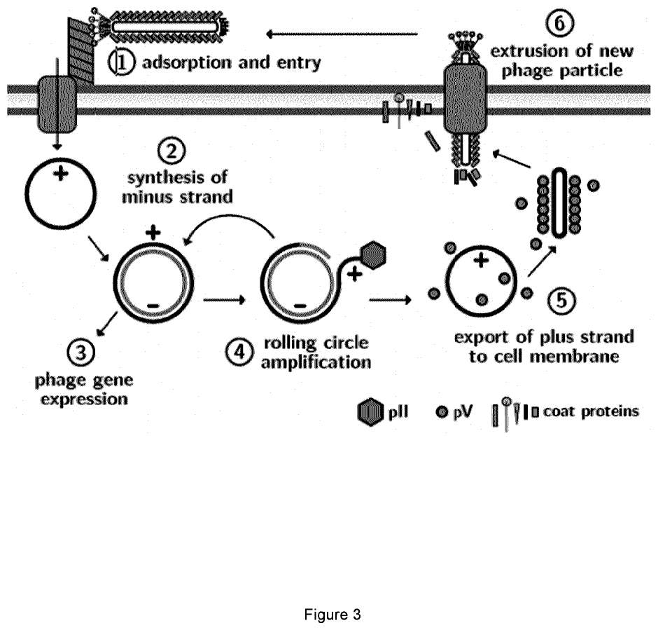

FIG. 3 shows a schematic of the lifecycle of a filamentous bacteriophage. The lifecycle of phage M13 (an example of a filamentous bacteriophage) can be broadly broken down into six steps: 1) M13 binds to the extended pilus via its minor coat protein, pIII. Retraction of the pilus brings the phage in contact with ToI proteins, through which it can enter the cell. 2) Upon cell entry, the plus single-stranded genome is converted into a double-stranded replicative form (RF) as host machinery synthesize the complementary minus strand using it as a template. 3) Phage genes are expressed. 4) The RF is replicated through a rolling circle mechanism of amplification, initiated by binding of phage protein pII. 5) Once pV levels increase and outcompete replication proteins, they bind the single-stranded plus strand and prepare it for export. 6) The phage particle is extruded as coat proteins assemble around the exported DNA molecule at the membrane.

Before phage replication can proceed, the single-stranded viral plus strand must be converted to a double-stranded episome, the RF. The complementary minus strand is synthesized by host proteins using the plus strand as a template. Both minus strand synthesis and phage replication are initiated within the intergenic region (IR). To synthesize the minus strand, E. coli RNA polymerase binds to the minus strand origin in the f1 on and generates a 20 nt RNA primer complementary to a stretch of nucleotides (positions 5736 to 5717 on the plus strand) of the minus strand. Extension from this primer by E. coli DNA polymerase Ill results in a double-stranded molecule (RF type IV) that is supercoiled by DNA gyrase to generate the RF type I (supercoiled RF). RFII refers to RF DNA nicked by phage proteins (open circular RF). After establishment of the supercoiled dsDNA episome, the newly synthesized minus strand of the RF serves as a template for phage gene expression. Expression of the replication protein pII is required before further phage replication can occur.

M13 replication proceeds through rolling circle amplification of the RF to generate more double-stranded RF. This process is initiated by phage protein pII and carried out by host replicative machinery. The f1 on is both necessary and sufficient for f1 phage replication as it contains the binding site for plI. A nicking-closing enzyme with topoisomerase I-like activity, pII initiates replication in two steps specifically at the f1 on of the plus strand in the RF.

Minivectors and bacteriophages encapsulating said minivectors are useful for delivering deoxyribonucleic acids to mammalian cells. Once delivered to the target cell the encapsulated minivector may be internalized by the target cell. In some embodiments, the deoxyribonucleic acid in the internalized minivector may then be expressed by the target cell machinery. The deoxyribonucleic acid may be used for any biological purpose that would benefit from the delivery of a deoxyribonucleic acid to a cell. For example, in an embodiment, the deoxyribonucleic acid to be delivered comprises a DNA vaccine. The delivery of the deoxyribonucleic acid may be useful for vaccination. In another example, the deoxyribonucleic acid may comprise an imaging agent or a diagnostic agent and may be used, for example, in the diagnosis of disease or in monitoring disease progression. The delivery of deoxyribonucleotides to a mammalian cell may be useful for the treatment or prevention of a disease or disorder. In an embodiment, the delivery of deoxyribonucleic acids to cells may be useful for tumor immunotherapy. In an embodiment, the deoxyribonucleic acid may be used for gene transfer or gene editing techniques and the delivery of such deoxyribonucleic acids would be useful for treating or preventing a disease or disorder in a subject in need thereof. The disease or disorder to be treated encompasses any disease or disorder that would benefit from the delivery of a deoxyribonucleic acid to a cell. The disclosed minivectors, bacteriophages encapsulating minivectors and compositions comprising encapsulated minviectrors may be used in methods of treating or preventing a disease or disorder in a subject in need thereof. In an embodiment, the disclosed minivectors are safe therapeutic vectors for delivery of deoxyribonucleic acids. In some embodiments, the deoxyribonucleic acid to be delivered comprises a target deoxyribonucleic acid which is meant to encompass a deoxyribonucleic acid sequence that is to be expressed in the target cell.

In an embodiment, a minivector may be used as a functional vector for rAAV production. A circular double-stranded DNA minivector may be collected and purified from target cells. In an embodiment, a (sense) mini replicative factor (RF) generated in host cells is a functional circular double-stranded DNA minivector.

In an embodiment, single-stranded DNA minivectors may be intracellular or phage-packaged circular ssDNA. In an embodiment, ssDNA minivectors can be purified from phage lysate. In an embodiment, ssDNA minivectors can be used for ssDNA applications such as aptamer production. In an embodiment, minivectors may be used for DNA origami.

In an embodiment, sense anti-sense circular ssDNA will form a mostly linear covalently closed (LCC) DNA minivector that can be purified from the host cell as a scalable LCC minivector prior to packaging by capsid proteins into a functional phagemid. In an embodiment, LCC DNA minivectors may be used as vectors for rAAV production, or mRNA production.

As used herein, “treat”, “treating”, “treatment of”, are used interchangeably. These terms refer to an approach for obtaining beneficial or desired results including but not limited to therapeutic benefit and/or a prophylactic benefit. By “therapeutic benefit” is meant eradication or amelioration of the underlying disease or disorder being treated. Also, a therapeutic benefit is achieved with the eradication or amelioration of one or more of the physiological symptoms associated with the underlying disorder such that an improvement is observed in the subject, notwithstanding that the patient is still afflicted with the underlying disorder. For prophylactic benefit, compositions comprising minivectors and bacteriophages are, in some embodiments, for administration to a subject at risk of developing a particular disease or condition, or to a subject reporting one or more of the physiological symptoms of a disease, even though a diagnosis of this disease has not been made.

The bacteriophage and minivectors may be present in a “therapeutically effective amount”, “effective amount” or “sufficient amount” which mean a quantity sufficient to achieve a desired result when administered to a subject. Therapeutically effective amounts may vary according to factors such as the age, sex, and weight of the subject. Dosage or treatment regimens may be adjusted to provide the optimum therapeutic response, as is understood by a skilled person.

In an embodiment, there is provided a pharmaceutical composition comprising a minivector as defined herein, a bacteriophage encapsulating the minivector as defined herein, a recombinant viral genome as defined herein, a phage particle as defined herein, or a targeted phage particle as defined herein; together with a pharmaceutically acceptable excipient, diluent, or carrier.

A “pharmaceutical composition” refers to a combination of ingredients that facilitates administration of one or more agents of interest (e.g. a therapeutic agent or a co-administrable agent) to an organism, e.g. a human or animal. A pharmaceutical composition generally comprises one or more agents of interest together with one or more pharmaceutically acceptable carriers or excipients. Many pharmaceutically-acceptable “carriers” or “excipients” are known in the art and these generally refer to pharmaceutically-acceptable materials, compositions, or vehicles, including liquid or solid fillers, diluents, excipients, solvents, binders, or encapsulating materials. Each component in the composition must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio. In one aspect, there is provided a composition comprising the minivector as defined herein, the bacteriophage encapsulating the minivector as defined herein, the recombinant viral genome as defined herein, the viral particle as defined herein, or the targeted viral particles as defined herein; together with a pharmaceutically acceptable excipient, diluent, or carrier.

### EXAMPLES

The following Examples outline embodiments of the invention and/or studies conducted pertaining to the invention. While the Examples are illustrative, the invention is in no way limited the following exemplified embodiments.

In the following Examples, E. coli K-12 strains were used in the generation of phage and plasmid constructs. E. coli JM109 was the host for plasmid amplification and purification unless otherwise specified. Bacterial strains were cultured in Luria-Bertani (LB) liquid medium, while plaque assays were carried out in top agar. Phage lysates were purified and stored in Tris-NaCl (TN) buffer. Where indicated, phage lysates were concentrated with polyethylene glycol. Bacterial strains, plasmids and bacteriophages used in the Examples are outlined in Tables 1 to 3 below.

TABLE 1
Bacterial strains.

| Strain | Genotype |
|---|---|
| JM109 | F′ traD36 proAB + lacI^{q} lacZ, ΔM15/Δ(lac-proAB) endA1 |
| | glnV44 thi-1 e14⁻ recA1 gyrA96 relA1 hsdR17 |
| XL1-Blue | F′ traD36 proAB + lacI^{q} lacZΔM15 Tn10/lac endA1 glnV44 |
| | thi-1 recA1 gyrA96 relA1 hsdR17 |
| Stbl4 | F′ proAB + lacI^{q} lacZΔM15 Tn10/endA1 glnV44 thi-1 recA1 |
| | gyrA96 relA1 Δ(lac-proAB) mcrA Δ(mcrBC-hsdRMS-mrr) |
| | λ-gal |
| NEB | F′ traD36 proAB⁺ lacI^{q} lacZΔM15/Δ(lac-proAB) glnV44 thi-1 |
| Turbo | galE15 galK16 R(zgb-210::Tn10)TetS endA1 fhuA2 Δ(mcrB- |
| | hsdSM)5(r⁻K, m⁻K) |
| ER2738 | F′ zzf::Tn10(TetR) proAB⁺ lacI^{q} lacZΔM15/Δ(lac-proAB) |
| | thi-1 glnV44, Δ(hsdS-mcrB)5, fhuA2 |
| DH5α | F⁻ φ80 Δ(lacZYA-argF)U169 endA1 thi-1 recA1 gyrA96 |
| | relA1 hsdR17(r⁻K, m⁻K) phoA supE44 |

TABLE 2
Plasmids

| Plasmid | Genotype |
|---|---|
| pGL2-SS-CMV-GFP- | pGL2-Promoter, cmv-gfp replaces SV40-luc, |
| SS | AmpR |
| pGL3-CMV | pGL3-Basic, cmv inserted in BlgII-HindIII, |
| | AmpR |
| pBluescript II KS+ | wildtype f1 ori, pUC ori, AmpR |
| pBR322 | AmpR, TetR |
| pSW9 | pBluescript II KS+, cmv-gfp from pGL2-SS- |
| | CMV-GFP-SS inserted in BamHI-EcoRI, |
| | AmpR |
| pSW10 | pBluescript II KS+, cmv-luc from pGL3- |
| | CMV inserted in KpnI, AmpR |
| pSW9-tet | pSW9, TetR from pBR322 inserted into Smal |
| pM13ori2 | split f1 ori inserted in pUC57 (Genbank |
| | Accession No. Y14837.1; M13 START and |
| | M13 STOP), AmpR |
| pM13ori2.cmvgfp | pM13ori2, cmv-gfp from pGL2-SS-CMV- |
| | GFP-SS inserted in EcoRI-PacI |
| pM13ori2.cmvluc | pM13ori2, cmv-luc from pGL3-CMV inserted |
| | in EcoRI-KpnI |
| pM13ori2.cmvgfp-tet | pM13ori2.cmvgfp, TetR from pBR322 |
| | inserted into KpnI |
| pPL451-gpD::egf | egf in translational fusion to gpD from |
| | λ in pPL451 (GN: AB248919.1) |
| pJET1.2/blunt | GN: EF694056.1 |
| pJET1.2-cmvgfp | pJET1.2, cmv-gfp inserted into EcoRV site, |
| | AmpR |
| pM13ori2.cmvgfp.SAS | pM13ori2.cmvgfp, second cmv-gfp inserted |
| (L) | in HindIII-BamHI |
| pM13ori2.cmvgfp.SAS | pM13ori2.cmvgfp, second cmv-gfp inserted |
| (NL) | in HindIII-BamHI |
| pM13ori2.cmvgfp.SAS | pM13ori2.cmvgfp, second cmv-gfp inserted |
| (GG) | via Golden Gate assembly |

TABLE 3
Bacteriophages

| Phage | Genotype |
|---|---|
| M13 | wildtype |
| M13KO7 | Tn903 (KanR), p15a ori |
| M13KE | M13, KpnI and EagI target sites in pIII |
| M13SW7 | M13KO7, gIII from M13KE |
| M13SW7-EGF | M13SW7, egf from pPL451-gpD::egf |
| M13SW8 | M13KO7, PS removed, KanR |
| full-(gfp) | cmv-gfp, from precursor pSW9, AmpR |
| full_{egf}-(gfp) | cmv-gfp, from precursor pSW9, pIII::EGF, AmpR |
| full-(luc) | cmv-gfp, from precursor pSW10, AmpR |
| full_{egf}-(luc) | cmv-gfp, from precursor pSW10, pIII::EGF, AmpR |
| mini-(gfp) | cmv-gfp, from precursor pM13ori2.cmvgfp, AmpR |
| mini_{egf}-(gfp) | cmv-gfp, from precursor pM13ori2.cmvgfp, |
| | pIII::EGF, AmpR |
| mini-(luc) | cmv-luc, from precursor pM13ori2.cmvluc, AmpR |
| mini_{egf}-(luc) | cmv-luc, from precursor pSW10, pIII::EGF, AmpR |
| mini-(gfp-tet) | from precursor pM13ori2.cmvgfp, AmpR, TetR |
| ds-mini-(gfp) | cmv-gfp − cmv-gfp, from precursor |
| | pM13ori2.cmvgfp.SAS (L) |

### Example 1

### Single Stranded DNA Minivectors

The regions of the f1 origin of replication required for initiation (M13 START) and termination (M13 STOP) identified by Short et al., 1988 (see FIG. 2) were inserted into a pUC57 backbone and separated by a polylinker. The cassette was synthesized and subcloned into pUC57 to construct pM13ori. The Bsal site present within the ampicillin resistance marker (bla) was replaced with Sphl through insertional mutagenesis to construct pM13ori2 in order to accommodate Bsal-dependent Golden Gate assembly. The deoxyribonucleotide sequence of minivector precursor, pM13ori2, is set forth in SEQ ID NO:1.

Expression cassettes comprising reporter genes (green fluorescent protein (GFP); cmv-gfp (2.1 kb); and luciferase; cmv-luc (2.6 kb)) were subcloned into the polylinker of pM13ori2 to generate mini-(gfp), and mini-(luc). To construct precursor vectors for “full” phagemid production, reporter gene expression cassettes were subcloned into the same wild-type f1 on backbone, pBluescript II KS+, to generate full-(gfp) and full-(luc). A fragment from pBR322 containing a fragment of the tet gene for TcR (1884 bp) was inserted into the precursor plasmid encoding cmv-gfp to form mini-(gfp)-(tet). The same fragment was also inserted into pSW9, the wild-type on phagemid, as the control pSW9-tet. The length of this final cmv-gfp-tet cassette was approximately 3.9 kb. The predicted length of the recombinant gfp-tet minivector is 4.4 knt, which is longer than the luc miniphagemid (3.1 knt). The purified final constructs were pM13ori2.cmvgfp-tet (cmv-gfp, tet), and pSW9-tet (cmv-gfp, tet). Both TcR constructs are approximately 2 kb larger than their non-TcR counterparts. The deoxyribonucleotide sequence of minivector precursor, pM13ori2cmvgfp, is set forth in SEQ ID NO:2. The deoxyribonucleotide sequence of minivector precursor, pM13ori2cmvluc, is set forth in SEQ ID NO:3.

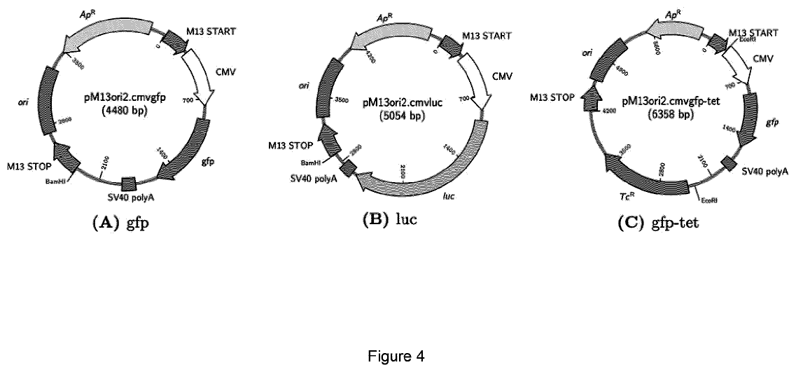

FIG. 4. Plasmid maps of exemplary minivector precursors. Reporter genes gfp (a) and luc (b) under the control of the universal promoter, CMV, were inserted between M13 START and M13 STOP in pM13ori2. A TcR fragment from pBR322 was inserted downstream of cmv-gfp to generate the third construct (c).

Amplification production and purification of phage, phagemids, and RF DNA. A fresh colony of the host carrying the minivector precursor was first inoculated into rich media and incubated at 37° C. with aeration until slightly turbid (0.01<A600<0.4). The culture was then infected with helper phage M13KO7 and returned to the same conditions for an additional 1-1.5 h. The culture was then supplemented with kanamycin (70 pg/mL) and returned to the same conditions overnight. The next day, the culture was centrifuged (8000×g, 10 min) to separate the bacterial pellet containing RF from the supernatant containing the phage lysate.

Purification of phage lysate. After removal of the cell pellet, the phage lysate was further purified through a 0.45 μm filter to remove residual bacterial debris. Filtered lysate was concentrated through precipitation with PEG: 1/5 of the lysate volume was added in PEG, and the mixture was incubated at 4° C. for at least 2 h. The lysate was then centrifuged at 12 000×g for 15 min at 4° C. to separate the PEGylated phage (pellet) from media (supernatant). The pellet was then re-suspended in a smaller volume of ice-cold TN buffer and a second PEG precipitation was carried out to further concentrate the phage. PEG-precipitated phage suspensions were treated with DNase I to remove any extraneous phage or bacterial DNA in the sample. The concentrated phage lysate was stored at 4° C.

Purification of the double-stranded RF and single-stranded minivector DNA. Episomal RF dsDNA were extracted from the pellet with the Monarch Plasmid Miniprep Kit. Phagemid DNA were extracted from the phage lysate through phenol-chloroform extraction. Phenol was added to PEG-concentrated phage lysate (1:1, v/v) and mixed by vortexing. After centrifugation for 5 min at 4° C., the top aqueous layer was extracted. This layer was then extracted again with an equivalent volume of phenol: chloroform twice, and chloroform once. Finally, the phagemid ssDNA was precipitated overnight with 100% ethanol at −80° C. The precipitated DNA was washed with 70% ethanol, dried, and re-suspended in RNase and DNase-free water.

Quantification of phagemid and RF DNA. Extracted DNA was analyzed on a NanoDrop 2000 to determine concentration and purity. Extracted RFs were linearized through digestion with BamHI and visualized via agarose gel electrophoresis (AGE), while phagemid ssDNA was visualized directly via AGE without digestion.

Quantification of phage lysates. The populations of helper and recombinant phagemids within each phage suspension were quantified using SYBR Green quantitative PCR (qPCR). Calibration curves for quantifying phage were constructed using external standards: M13KE RF (7222 bp) for helper phage, pGL2-SS-CMV-GFP-SS (5257 bp) for gfp-encoding target phage, and pGL3-CMV (5678 bp) for luc-encoding target phage. Primers specific to a region within g5 of M13KO7 were used to amplify helper phage. Primers targeting a region within gfp or luc were designed to amplify gfp- or luc-encoding phage. To generate each calibration curve, 10-fold serial dilutions of each external control were prepared (10-1 to 10⁻⁸) as template for the PCR reaction.

A plaque assay was conducted to quantify viable phage. Plaque and colony assays used ER2738. A colony assay was conducted to quantify the ability of infectious helper phage to confer antibiotic resistance.

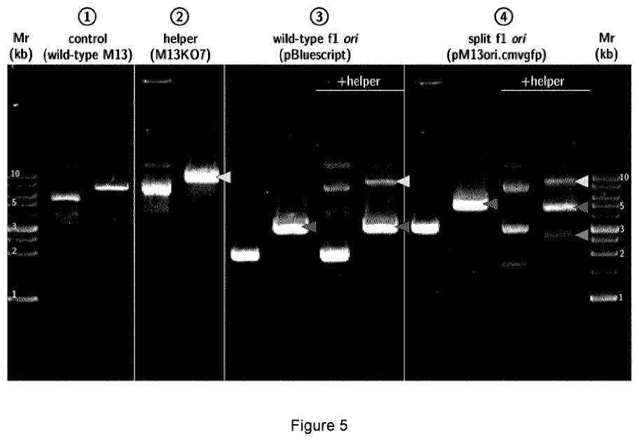

FIG. 5 shows the production of recombinant RFx species from precursor vectors. Helper phage M13KO7 produced a new recombinant replicative form (RFx) from the minivector precursor, and packaged the recombinant species into progeny phage particles. Purified RF dsDNA was linearized with BamHI and visualized via AGE. RF DNA isolated from cells infected with wild-type M13 or helper phage M13KO7 alone resolved to their respective genome sizes: 6:4 kb and 8:7 kb. Two expected RF DNA species were present in helper-infected cells transformed by pBluescript II KS+: the helper phage genome and the phagemid (3 kb). In cells transformed by the minivector precursor, pM13ori2.cmvgfp, a single band corresponding to the expected plasmid size (4:5 kb) was observed after BamHI digestion. An additional DNA species was observed only in the presence of helper phage, which was approximately the expected size for the RFx molecule (2:6 kb).

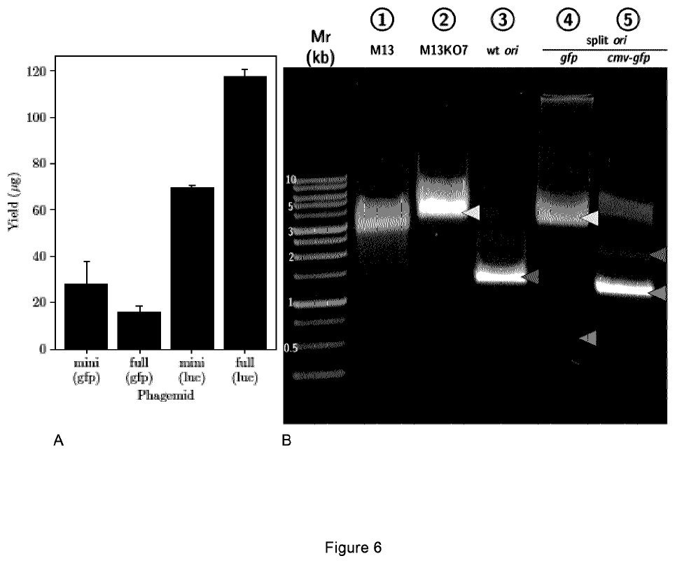

FIG. 6 shows DNA isolated from phage lysates. (A) Purified ssDNA was quantified on Nanodrop and compared across different phagemids. (B) The ssDNA was also visualized on AGE, alongside a dsDNA ladder. The ssDNA runs at approximately half its expected dsDNA size. From left to right, lysates were prepared from: 1) wild-type M13 (6:4 knt), 2) M13KO7 alone (8:7 knt), 3) pBluescript II KS+(3 knt), 4) pM13ori2 with an approximately 1 kb fragment of gfp inserted, 5) pM13ori2.cmvgfp (plasmid: 4:5 knt, miniphagemid: 2:2 knt). Arrows indicate bands of interest: the top two arrows in lanes 2 and 4 indicate the helper genome, the middle two arrows overall (the top arrows in lanes 3 and 5) identify the plasmid or full phagemid, and the lower two arrows in lanes 4 and 5 indicate single stranded minivector DNA. For wild-type M13 and helper phage alone, a single ssDNA species was observed. For M13KO7-assembled pBluescript II KS+, a single band corresponding approximately to the expected phagemid size (3 knt) was observed.

Alongside pM13ori2.cmvgfp, an intermediate precursor was also constructed with a 1 kb gfp sequence. Two distinct phage species were observed in the ssDNA: the larger band corresponds to the size of helper phage, while the smaller band corresponds to the expected size of the minivector. Three bands were observed from mini-(gfp) that were the expected sizes for the helper genome (8.7 knt), the minivector precursor (4.5 knt), and a third band corresponding to the expected size of recombinant miniphagemid.

Quantification of the ssDNA from phage lysates showed that yield was not impacted by whether the phagemid carried a split or intact f1 origin of replication. As measured by Nanodrop, the yield of single-stranded DNA from the phage lysate was comparable across both wild-type and split origin of replication. No significant difference was observed in the yield of ssDNA arising from a split f1 origin of replication compared to an “intact” one for cmv-gfp, although more full-(luc) ssDNA was produced than mini-(luc). Conversion from mass to number of molecules shows that the ssDNA yields are comparable: 2.116×10¹³ for mini-(gfp), 1.077×10¹³ for full-(gfp), 4.322×10¹³ for mini-(luc) and 4.088×10¹³ for full-(luc).

### Production Efficiency of Miniphagemids and Full Phagemids

DNA quantification approaches such as qPCR have been established as highly accurate methods for the quantification of viral genomes, which is equivalent to counting the number of virion particles since every virion encapsulates one phage genome. To estimate phage concentration, calibration curves were generated from a dilution series of each exogenous plasmid control: M13KE (g5), pGL2-SS-cmv-gfp-SS (gfp), and pGL3-cmv (luc).

Concentrations determined through qPCR were compared against those determined through plaque assay and colony assay. Since M13KO7 encodes a selectable marker, it was possible to quantify M13K07-infected cells that gain resistance to kanamycin. The titre of plaque or colony forming units as estimated were compared against the concentrations of total and helper phage only as estimated by qPCR. For clarity, the quantification by plaque or colony assay was reported as the phage titre, with units PFU or CFU per volume, as it refers to the number of viable or infectious phage. Quantification by qPCR is reported as the virion or phage concentration, with units of genome copies (gc) per volume.

Plaque titre estimated by plaque or colony assay was compared to qPCR. For the heterogeneous lysates comprised of both phagemid and helper, the plaque and colony assay-estimated titres were not significantly different from each other, but were both greater than the qPCR-estimated virion concentration. The qPCR-estimated helper concentration of the mini-(gfp) lysate was statistically significantly lower than the titre as estimated by colony assay (p<0.05). In contrast, the qPCR-estimated helper concentration of the full-(gfp) lysate was statistically significantly lower than the titre as estimated by plaque assay (p<0.05). It was also lower than the colony assay-estimated titre, but this was above the significance level (0.05<p<0.1).

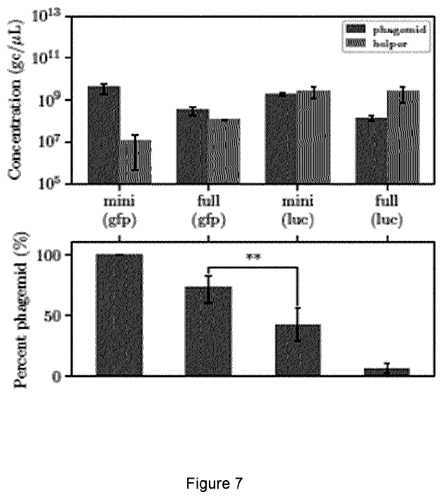

FIG. 7 summarizes the virion concentration for each minivector or full vector phagemid and helper phage per lysate in the top panel. In the bottom panel, the fraction that the target phagemid comprises of the overall lysate is shown as a percentage for each sample. Both phage species were highly abundant in the PEGylated samples, with no phage species present at concentrations below 10⁶ gc/pL. Total virion concentration (including both helper and target phagemid) is reported in Table 4. No significant difference in overall yield was observed.

TABLE 4
Comparison of phage yield from minivector
and full vector phagemids

Total virion concentration (×10⁸ gc/μL)

| Strain | mini | Full |
|---|---|---|
| cmv-gfp | 38.49 ± 19.82 | 4.36 ± 1.32 |
| cmv-luc | 45.51 ± 13.72 | 25.87 ± 16.80 |

The composition of phage lysates appeared to depend on the encoded cassette and whether the origin was split or intact. The phagemid fraction decreased between the minivector and full vector-containing lysates for each respective transgene: mini-(gfp) comprised 99.7% of the lysate while full-(gfp) comprised 73.2%. Similarly, mini-(luc) comprised 42.4% while full-(luc) comprised only 5.8%. Differences in composition were all statistically significant (p<0.05), except for the difference between full-(gfp) and mini-(luc) (0.05<p<0.1).

M13KO7 preferentially packages itself over the cmv-luc phagemids, while the cmv-gfp encoding phagemids appear to be preferentially packaged. The cmv-luc transgene cassette is longer than the cmv-gfp cassette and the full phagemids are longer than their minivector counterparts. The shortest miniphagemid, mini-(gfp) (2.5 knt), was produced at near complete efficiency, with helper phage comprising less than 1% of the final lysate. Similarly, a decrease in packaging efficiency was observed between the longest phagemid, full-(luc) (5.9 knt) compared with its mini-counterpart (mini-(luc) at 3.1 knt). However, while full-(gfp) (5 knt) is longer than mini-(luc), full-(gfp) may be packaged with comparable or better efficiency (0.05<p<0.1).

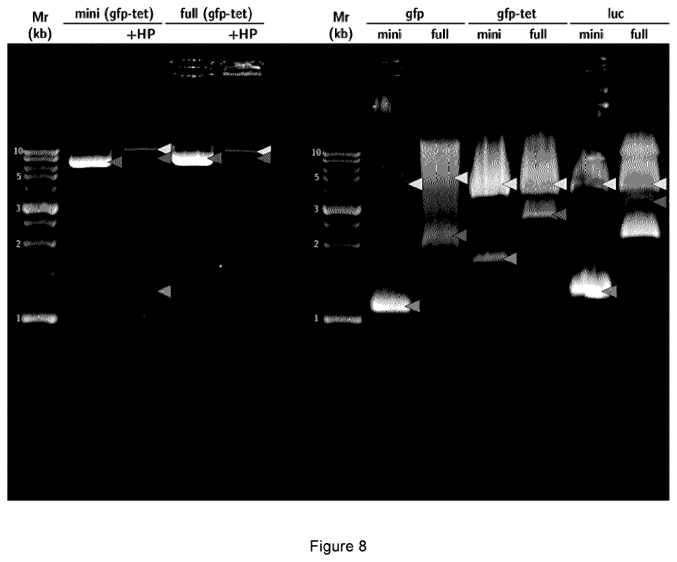

FIG. 8. Examination of Pacl-linearized RF DNA on AGE revealed faint bands approximately around the sizes of the expected RFx, although bands of other sizes are also visible. Production of the miniphagemid mini-(gfp-tet) and its wild-type on counterpart full-(gfp-tet) was observed by visualizing purified phagemid ssDNA alongside that of the mini and full phagemids for the other two cassettes, cmv-gfp and cmv-luc. Single-stranded DNA runs at approximately half the size expected for its dsDNA counterpart, when referring to a double-stranded ladder. Helper phage genome is visible across all lanes for purified ssDNA, indicated by the uppermost arrows. Distinctive bands corresponding to the minivectors and full vectors were also observed, marked by the four lowermost arrows. Full-(luc) showed multiple bands, indicating some recombinant secondary species present in the lysate.

Overall virion concentrations are shown in Table 5. The total virion concentration of full-(gfp) significantly increased during these culture conditions compared to the previous conditions, while the total concentration of mini-(luc) significantly decreased (p<0.05). Overall yield was not found to differ significantly between lysates in this cohort.

TABLE 5
Comparison of phage yield across different phagemid cassettes

Total virion concentration (×10⁸ gc/μL)

| Strain | mini | full |
|---|---|---|
| cmv-gfp | 5062.13 ± 6562.92 | 3372.98 ± 1276.59 |
| cmv-luc | 0.71 ± 0.68 | 207.71 ± 413.69 |
| cmv-gfp-tet | 860.52 ± 902.79 | 305.38 ± 313.02 |

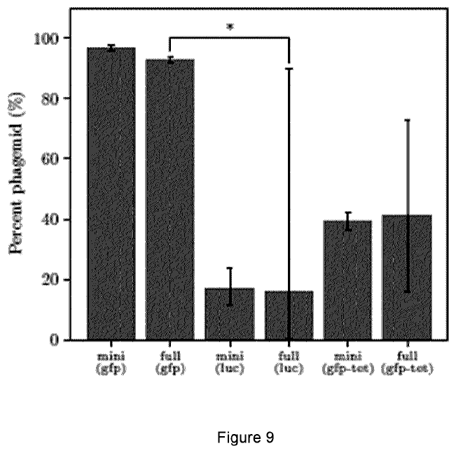

FIG. 9 shows the composition of phage lysates encoding one of cmv-gfp, cmv-luc, or cmv-gfp-tet. The proportion of the target phagemid is shown as a percentage of the total phage population. Error bars represent SEM×2, n=3. The * above the bars indicates a difference at significance level p<0.05. Quantification of (gfp), (luc), and (gfp-tet) phagemid lysates also revealed high ratios of mini-(gfp) and full-(gfp) phagemid to helper phage and much lower ratios of mini-(luc) and full-(luc). The mini-(gfp) (96.9%) and full-(gfp) (93.0%) phagemid fractions were higher than both their (gfp-tet) and (luc) counterparts (p<0.05). Under these culture conditions, no significant difference was observed between the mini and full phagemid fractions for cmv-gfp (p>0.05). The mini-(gfp-tet) phagemid comprised only 39.2% of its lysate while the full-(gfp-tet) comprised 41.5%. The cmv-luc phagemids again comprised the smallest fractions: 16.7% for mini-(luc) and 16.1% for full-(luc). Although the length of the cmv-gfp-tet cassette was much longer than cmv-luc, no significant difference was observed between their lysate compositions (p>0.05).

Miniphagemids were Preferentially Assembled Over Full Vector Phagemid Vectors

Surprisingly, the split on was not observed to impact overall yields nor compromise phagemid production. Full vector phagemids comprised a smaller fraction of lysates than their minivector-counterparts. It was expected, based on the observations by Short et al., 1988, that the intact f1 origin on wild-type phagemid controls (pSW9, pSW10, pSW9-tet) would be preferentially replicated and packaged over the helper phage genome. The split origin (pM13ori2.cmvgfp, pM13ori2.cmvluc, pM13ori2.cmvgfp-tet) was expected to be less efficiently processed, as the intact f1 origin first needs to be formed.

It was observed that the helper phage preferentially assembled itself over a cmv-luc-encoding phagemid, regardless if the origin of the target phagemid was split or wild-type. A longer transgene cassette was constructed through the introduction of a pBR322 fragment into the cmv-gfp cassette, leading to a final cassette size of 3.9 kb and final phagemid size of 4.4 knt. If phagemid length size did affect production efficiency, mini-(gfp-tet) and especially full-(gfp-tet) (6.9 knt) would be packaged even less efficiently than mini-(luc) (3.1 knt) and full-(luc) (5.9 knt). The mini-phagemid and full-(gfp) phagemid fractions were much higher than their (luc) and (gfp-tet) counterparts; in contrast to the PEGylated lysates, no significant difference was observed in these lysate compositions. No significant difference in lysate composition was observed between the (luc) and (gfp-tet) phagemids despite their large differences in phagemid length. Notably, greater variance in composition was observed for both full-(luc) and full-(gfp-tet) under these culturing conditions.

The replication and assembly of miniphagemid from minivector precursors encoding transgenes of interest flanked by separated regions of the M13 f1 on was shown. The assembly of a recombinant minivector from a split f1 on was superior to assembly of the same transgene cassette in a backbone containing a wild-type f1 ori. High titres of M13 compensated for low production of any of the phagemids under study. For example, although full-(luc) comprises only a small fraction of a PEGylated phage lysate, well over 108 virions are present.

### Example 2

### Targeted Helper Phage Rescue of Miniphagemid

In this Example, a derivative of M13KO7 was constructed for Type 3 display of a mammalian ligand, epidermal growth factor (EGF), for use in mammalian cell targeting. When used as a cell-specific targeting ligand, EGF recognition by its cognate receptor has been shown to enable phage internalization into mammalian cells, which can then enable phage-mediated gene transfer in a tissue-specific manner (Larocca et al., 2001). Mono- and multivalent display of EGF has previously been demonstrated on M13; however, EGF-displaying helper rescue of phagemid has not been demonstrated to the inventor's knowledge. The 53-residue EGF peptide is amenable to display on filamentous phage despite its length as it is not majorly hydrophobic, nor very cationic. Minivectors expressing reporter genes gfp and luc under control of the strong universal promoter cmv, including those from Example 1, were packaged using helper phage with multivalent EGF display.

Construction of an M13KO7 derivative capable of display on pIII. M13KE has endonuclease target sites in gIII that simplify N-terminal peptide fusions, while M13KO7 contains the plasmid p15a on for phage-independent amplification and a KmR marker. The gIII from M13KE was inserted into the helper phage M13KO7 using Gibson assembly in order to generate a helper phage that could easily take on N-terminal pIII fusions while retaining the p15a ori. The M13KO7 backbone was amplified with primers extending outward from its gIII region, excluding gIII itself: M13KO7-F and M13KO7-R. Primers had overlapping regions for assembly. Amplified PCR fragments were mixed with NEBuilder HiFi DNA Assembly Master Mix for assembly, transformed into DH5a, and selected on LB agar supplemented with 25 g kanamycin. The purified final construct, M13SW7, was transformed into an F+E. coli strain for further characterization.

Construction of a helper phage displaying EGF (M13SW7). The display peptide EGF was inserted as an N-terminal fusion to pIII after the peptide leader sequence, via GGGS (Gly-Gly-Gly-Ser), a flexible linker used to join components of fusion proteins. Primers were designed to amplify EGF from pPL451-gpD-EGF. In the forward primer (Kpnl-egf-F), the gIII leader sequence and Kpnl target site were part of the 5′ primer overhang. In the reverse primer (Eagl-egf-R), the linker and the Eagl target site were part of the 3′ overhang. The amplified PCR fragment and the target phage genome, M13SW7, were digested with Kpnl and Eagl. Purified digested fragments were ligated with T4 DNA ligase, transformed into XL1-Blue, then selected on LB agar supplemented with kanamycin. The purified final construct was M13SW7-EGF. Successful display of the peptide was verified through immunoassays, including both ELISA and dot blot. Signal was only detected for M13SW7-EGF and not M13SW7, confirming the presence of the EGF ligand.

Phage amplification. To produce helper phage, a fresh colony of E. coli XL1-Blue was inoculated into 2×YT supplemented with MgSO₄, and incubated at 37° C. with agitation until slightly turbid (0.05<A600<0.4). Helper phage was then added until a final phage titre of approximately 10⁸ PFU/mL and returned to the same conditions for an additional 1.5 h. The culture was then supplemented with kanamycin (final concentration: 70 μg/mL) and returned to the same conditions overnight. The next day, the culture was centrifuged (8000×g, 10 min) to separate the bacterial pellet containing RF from the supernatant containing the phage lysate.

Purification of Phage lysate. After removal of the cell pellet, the phage lysate was further purified through a 0.45 μm filter to remove residual bacterial debris. Filtered lysate was concentrated through precipitation with PEG: 1/5 of the lysate volume was added in PEG, and the mixture was incubated at 4° C. for at least 2 h. The lysate was then centrifuged at 12 000×g for 15 min at 4° C. to separate the PEGylated phage (pellet) from media (supernatant). The pellet was then re-suspended in a smaller volume of ice-cold TN buffer and a second PEG precipitation was carried out to further concentrate the phage. PEG-precipitated phage suspensions were treated with DNase I to remove any extraneous phage or bacterial DNA in the sample. The concentrated phage lysate was stored at 4° C.

Purification of the double-stranded RF and single-stranded minivector DNA. Episomal RF dsDNA were extracted from the pellet with the Monarch Plasmid Miniprep Kit. Phagemid DNA were extracted from the phage lysate through phenol-chloroform extraction. Phenol was added to PEG-concentrated phage lysate (1:1, v/v) and mixed by vortexing. After centrifugation for 5 min at 4° C., the top aqueous layer was extracted. This layer was then extracted again with an equivalent volume of phenol:chloroform twice, and chloroform once. Finally, the phagemid ssDNA was precipitated overnight with 100% ethanol at −80° C. The precipitated DNA was washed with 70% ethanol, dried, and re-suspended in RNase and DNase-free water. Extracted DNA was analyzed on a NanoDrop 2000 to determine concentration and purity. Extracted RFs were linearized through digestion with BamHI and visualized via agarose gel electrophoresis (AGE), while phagemid ssDNA was visualized directly via AGE without digestion.

Confirmation of EGF display was done by Dot blot analysis comparing M13SW7-EGF against purified recombinant EGF and by semi-quantitative ELISA, again comparing M13SW7-EGF against purified recombinant EGF.

A culture of cells with both the helper phage and target phagemid vector was grown overnight in 2×YT supplemented with MgSO₄, kanamycin, and ampicillin. Phage lysates were purified from these cultures the next day and concentrated through PEG precipitation. These phage lysates were incubated with early log-phase E. coli ER2738 (A600 ˜0.4) and spotted on LB agar plates supplemented with ampicillin, kanamycin, or both. For full plates, the same procedure was followed except that the entire dilution volume (200 μL) was spread on an LB agar plate supplemented with ampicillin, kanamycin, or both. The titre was determined from the mean of three counts and expressed as colony forming units (CFU) per millilitre.

Quantification of phage species. The populations of helper and recombinant phagemids within each phage suspension were quantified through SYBR Green quantitative PCR (qPCR). Calibration curves for quantifying phage were constructed using external standards: M13KE RF (7222 bp; New England BioLabs) for helper phage, pGL2-SS-CMV-GFP-SS (5257 bp; Mediphage Bioceuticals) for gfp-encoding target phage, and pGL3-CMV (5678 bp) for luc-encoding target phage. Primers specific to a region within g5 of M13KO7 were used to amplify helper phage. Primers targeting a region within gfp or luc were designed to amplify gfp- or luc-encoding phage. To generate each calibration curve, 10-fold serial dilutions of each external control were prepared (10⁻¹ to 10⁻⁸) as template for the polymerase chain reaction (PCR) reaction.

To prepare the phage particles for PCR, the lysates were denatured by heat for 100° C. for 15 min to isolate phage DNA. Ten-fold serial dilutions of each cleared lysate were prepared as template for the PCR reaction. As the phage lysates were of unknown concentration, dilution series of each were amplified through qPCR. For PEGylated lysates, dilutions in the range 10⁻³-10⁻⁵ were examined, while for non-PEGylated lysates, 10⁻¹-10⁻³.

Each 10 μL PCR reaction was prepared using 5 1 μL of PowerUp SYBR Green Mix (Thermo Fisher Scientific, Waltham, USA), 1 1 μL each of 500 nM primer (forward and reverse), 2 1 μL of template, and 1 1 μL of dH₂O. PCR cycling conditions were as follows: 50° C. for 2 min, 95° C. for 2 min, followed by 40 cycles at 95° C. for 15 s and 60° C. for 1 min. Next, the melt curve was set for 1 cycle at 95° C. for 15 s, 60° C. for 1 min, and 95° C. for 15 s. PCR reactions were run in triplicate on the StepOne Plus Real-Time PCR system (Applied Biosystems, Waltham, USA). The quantification cycle or threshold cycle number (Cq) for each reaction was recorded and used in subsequent analysis.

Conversion from the mass of dsDNA to the number of genome copies is given by

$$gc = ( \frac{mass}{size} \times 607.4 + 157.9 ) \times ( 6.02 \times 1 {0}^{23} )$$

where gc is the concentration of phage genome copies (genome copies/μL), mass is the mass the dsDNA standard in g/μL, and size is its length in bp.

Phage concentrations were estimated from their respective calibration curve. First, the Cq values from each exogenous control are plotted against the log of the known concentrations for each concentration in the dilution series. Linear regression produces a familiar equation of the form Cq=mx+b, where Cq is the measured threshold cycle number, m is the slope, x is the base-10 log of the concentration in gc/μL and b is the x-intercept. PCR amplification efficiency is given by E=10^{-1/m}−1.

Subsequently, virion concentration V were estimated simply by V=10^{(Cq-b)}/m×2, where multiplication by 2 adjusts for the estimation of single-stranded products (gc/μL) from dsDNA standards. Only Cq measurements within the bounds of the calibration curve were used to calculate phage concentration.

Phagemid fractions were determined as the concentration of target phagemid divided by the total virion concentration, expressed as percentages. As compositional data, they have a fixed constant sum constraint (100%). In order not to violate this constraint, the data were transformed using an isometric log ratio transformation before performing statistical analyses. Data were then transformed back to percentages for reporting.

### Characterization of EGF-Displaying Helper Performance

Phage tolerance of the fusion was assessed by plaque assay of PEGylated lysates prepared from 50 mL of culture (Table 6). The lowest titres were from wild-type M13 and M13KE. M13SW7-EGF titres were comparable to those of its predecessors, M13SW7 and M13KO7. Thus, the EGF fusion did not impede phage infectivity.

TABLE 6
Phage titre of the EGF-displaying helper phage

| Phage | Titre (×10¹³ gc/μL) |
|---|---|
| M13 | 0.31 ± 0.06 |
| M13KO7 | 1.90 ± 0.12 |
| M13KE | 0.12 ± 0.02 |
| M13SW7 | 3.43 ± 1.73 |
| M13SW7-EGF | 2.75 1.04 |

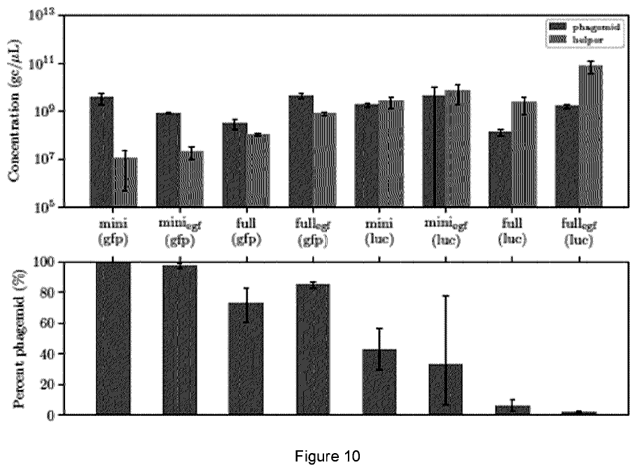

FIG. 10 shows the ability of M13SW7-EGF to assemble miniphagemid and phagemid from split and wild-type origins. The overall yield as well as the minivector:helper ratios were compared between M13SW7-EGF and M13KO7. At the top of FIG. 9 the concentration (gc=μL) of each phage species (target phagemid or helper) in each lysate is presented. Below, the phagemid fraction of each phage species is shown as a percentage of the total phage population.

Yields from lysates prepared using M13SW7-EGF are shown in Table 7. The yield of full-egf-(luc) was the highest overall; it was significantly higher than both its non-displaying counterpart, full-(luc), and its mini counterpart, mini-egf-(luc) (p<0.05). As observed in Example 1, the cmv-gfp encoding phagemids were produced at a higher phagemid:helper ratio than cmv-luc. No significant difference in phagemid compositions was observed between the use of M13KO7 or M13SW7-EGF as the helper. Both mini-(gfp) and mini-egf-(gfp) comprised the largest fractions of their lysates, followed by full-(gfp) and full-egf-(gfp). Both mini-(luc) and mini-egf-(luc) performed better than full-(luc) and full egffull-egf-(luc), which comprised the smallest fractions of their lysates.

TABLE 7
Comparison of phage yield from the EGF-displaying helper

| Total phage concentration (×10⁸ gc/μL)¹ | |
|---|---|
| mini | full |

| Cell line | −EGF | +EGF | −EGF | +EGF |
|---|---|---|---|---|
| cmv-gfp | 38.49 ± | 8.56 ± | 4.36 ± | 54.56 ± |
| | 19.82 | 0.33 | 1.32 | 10.84 |
| cmv-luc | 45.51 ± | 118.81 ± | 25.87 ± | 819.78 ± |
| | 13.72 | 39.42 | 16.80 | 430.10 |

A pIII display helper system for the packaging of phagemids has been demonstrated. No impact from M13 pIII display of EGF on phage viability, nor ligand recognition by its cognate antibody was observed. The M13SW7 and M13SW7-EGF titres were among the highest of the group.

M13KO7 was modified to display a mammalian cell-specific ligand, EGF. The resulting phage, M13SW7-EGF, maintained comparable infectivity to its parent phages and was able to process split origin precursor plasmids into minivectors as efficiently as M13KO7.

### Example 3

### PS-Deficient Helper Phage

A derivative of M13KO7 was constructed wherein the packaging signal (PS) directly downstream of gIV and upstream of Tn903 was deleted and replaced with a Rho-independent terminator to generate M13SW8. The deoxyribonucleic acid sequence of M13SW8 is set forth in SEQ ID NO:4.

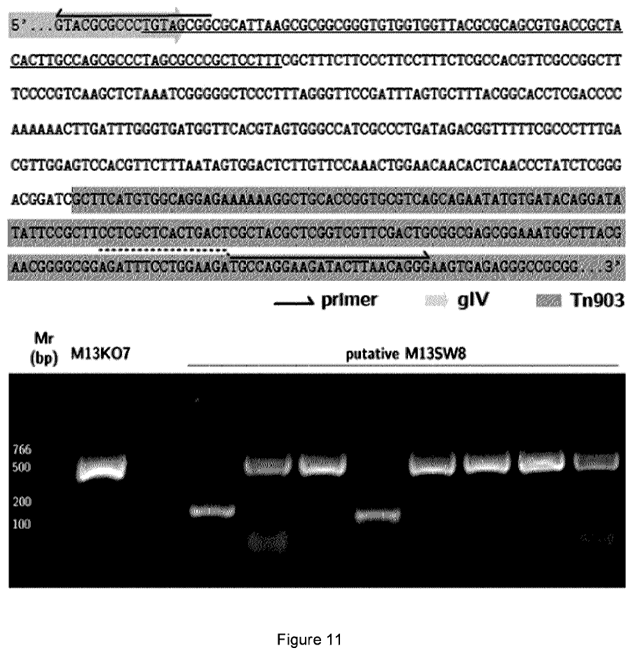

In FIG. 11, the region between gIV and Tn903 in M13KO7 is shown. Nucleotides identified as part of the PS loop are underlined. Primers for mutagenesis and relevant features are depicted. In the bottom panel PCR products were visualized using AGE and bands of the expected sizes were observed. Deletion of the PS prohibits phage genome interaction with phage assembly complexes, thereby preventing extrusion of the helper; the loss of PS has been linked to reduced phage particle production.

PS deficient bacteriophage reduced helper phage contamination in phagemid lysates thereby improving minivector rescue. In other words, the absence of a PS on the helper phage led to preferential extrusion of phagemid progeny over the helper and improved the phagemid:helper ratio. Methods including phage purification and DNA purification were performed as described in Example 2.

M13KO7 and M13SW8 phage were propagated in ER2738 alone and in presence of pBluescript II KS+. Phage lysates were purified and concentrated via precipitation with PEG. Plaque formation by spots of M13KO7 lysate on a lawn of ER2738 were seen, but no plaques even at low dilutions for M13SW8. Notably, attempts to extract ssDNA from M13SW8 lysates were unsuccessful. Absence of plaque formation and ssDNA in the lysate suggest that there was no M13SW8 phage production.

Lysates of pBluescript II KS+ phagemid assembled by M13KO7 or M13SW8 were incubated with susceptible ER2738. The infected cultures were spotted onto LB agar supplemented with ampicillin or ampicillin and kanamycin. Ampicillin resistance arose by cell uptake of ApR pBluescript phagemid particles; single colonies could not be discerned even at high dilutions (10⁻⁸), indicating a high phagemid titre from both helpers. Kanamycin resistance also emerged by cell uptake of KmR M13KO7 or M13SW8 helper phage particles. No ApRKmR colonies were visible past the 10⁵ dilution spot for the M13SW8-packaged lysate. In comparison, single colonies could not be discerned for the M13KO7-packaged lysate, indicating much higher concentration of M13KO7 in the phagemid lysate compared to M13SW8.

Titres determined from helper phage or helper-rescued pBluescript lysates show similar results (Table 8). Consistent with the observations from the spot plates, it was seen that while the lysate generated from M13SW8-rescue of pBluescript exhibited a tenfold higher ApR colony titre than M13KO7-rescued lysate (3.00×10¹⁴ versus 4.37×10¹³ CFU/mL), the Km^{R} colony titre was much lower. The titre of the Km^{R} phage (M13SW8) is seven orders of magnitude lower than that of the Ap^{R} phage (phagemid) in the M13-rescued lysate. In comparison, the titres of Ap^{R} and Km^{R} phage are much more similar when M13KO7 is the helper.

TABLE 8
Phage Titres from a helper deficient of its packaging signal

| | |
|---|---|
| M13KO7 | M13SW8 |

Phage only

| | | |
|---|---|---|
| PA | 3.16 × 10¹³ | — |
| CA (Km) | 2.61 × 10¹⁴ | — |

Rescue of pBluescript II KS+

| | | |
|---|---|---|
| CA (Ap) | 4.37 × 10¹³ | 3.00 × 10¹⁴ |
| CA (Km) | 4.43 × 10¹³ | 3.93 × 10⁷ |
| CA (Ap/Km) | 4.53 × 10¹³ | 3.94 × 10⁷ |

PA: Plaque assay (PFU/mL); CA: Colony assay (CFU/mL), antibiotic indicated in parentheses.

### Characterization of the Performance of Self-Packaging Deficient Helper Phage

The efficiency of M13KO7 and M13SW8 in the production of mini-(gfp) and full-(gfp) was compared across six strains that represent common laboratory strains for plasmid or M13 propagation. The five F+ strains were JM109, XL1-Blue, Stbl4, NEB Turbo, and ER2738; an F− control, DH5a, was also compared. Each strain was transformed by the miniphagemid precursor pM13ori2.cmvgfp or the full phagemid plasmid (pSW9) and either M13KO7 or M13SW8 to act as the helper. Total phage concentrations are given in Table 9 while phagemid fractions for each lysate are shown in FIG. 11.

TABLE 9
Comparison of phage yield by two helper
phages across different strains

| Total phage concentration (×10⁸ gc/μL) | |
|---|---|
| M13KO7 | M13SW8 |

| Strain | mini | full | mini | full |
|---|---|---|---|---|
| JM109 | 2.33 ± | 5.24 ± | 5.08 ± | 8.33 ± |
| | 0.47 | 3.46 | 0.21 | 2.94 |
| XL1-Blue | 2.32 ± | 6.61 ± | 1.29 ± | 2.78 ± |
| | 0.36 | 2.69 | 0.55 | 0.67 |
| Stb14 | 13.93 ± | 11.77 ± | 0.80 ± | 8.98 ± |
| | 5.28 | 3.62 | 0.40 | 3.34 |
| NEB Turbo | 1.01 ± | 1.10 ± | 0.78 ± | 0.42 ± |
| | 0.04 | 0.34 | 0.38 | 0.10 |
| ER2738 | 1.38 ± | 6.85 ± | 0.69 ± | 3.85 ± |
| | 0.35 | 2.59 | 0.18 | 2.12 |
| DH5α | 17.47 ± | 14.72 ± | 1.10 ± | 55.38 ± |
| | 16.58 | 8.18 | 0.58 | 47.05 |

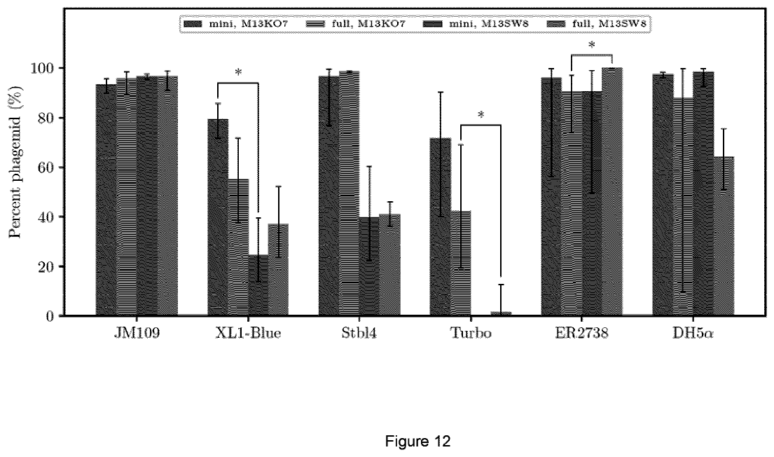

FIG. 12 shows the proportion of packaged target phagemid (either mini-(gfp) or full-(gfp)) as a percentage of the total phage population, as produced by either M13KO7 or M13SW8 (PS-deficient helper phage) across 6 different E. coli strains. The * above the bars indicates a difference at significance level p<0.05. The host background more strongly impacted phagemid production than the choice of helper.

Note that cells were transformed by helper phage RF DNA in this study instead of being directly infected as in Example 1. No difference in helper phage activity was observed between these two methods of DNA delivery.

Total phage titres using M13KO7 as the helper did not differ significantly; however a higher total phage titre was observed in M13SW8 rescue of full-(gfp) in JM109 when compared to all other strains (p<0.05). Both M13KO7 and M13SW8 were able to rescue phagemid in DH5a, despite the lack of an F′ episome. In fact, some of the highest titres were from DH5a, up to 5.54×10⁹ gc/μL.

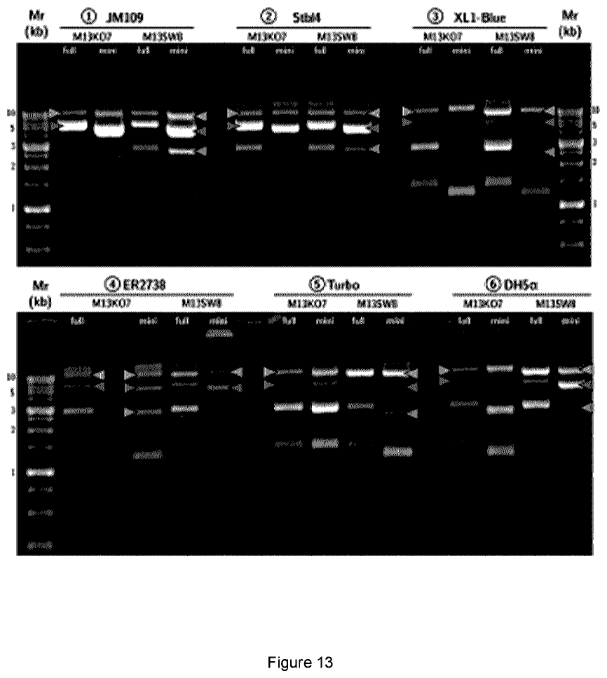

FIG. 13 shows that PS-deficient helper phage produce recombinant RF (RFx) in 6 E. coli strains. M13SW8 and M13KO7 helper activity was evaluated with respect to cmv-gfp encoding phagemids across different host backgrounds.

PS-deficient helper phage improves the phagemid:helper ratio. The effect is more pronounced in a recA, endA strain such as JM109. The strain-helper combination of JM109 and M13SW8 was a good combination for minivector production.

### Example 4

### Delivery of Minivector Particles to Mammalian Cells

Purified single-stranded minivector DNA, full phagemid vector DNA, and encapsulated phage particles were administered across multiple mammalian cell lines including HeLa, HEK293T, HT-29, MRC-5 and A549 which represent different tissue types. Miniphagemids lacking a prokaryotic bacterial/phage backbone improved DNA transfer in mammalian cells compared to their wild-type origin (full vector) phagemid counterparts.

Phage particles displaying the cell-targeting ligand, EGF, improved cellular uptake and subsequent DNA transfer in EGFR-expressing cells in contrast to phage without any display. Cell receptor targeting improved the uptake and subsequent DNA delivery to the target cell. Complexation with a commercial cationic lipid transfection reagent improved the delivery of phage particles to mammalian cells.

Plasmids and double-stranded replicative factors (RFs) were extracted and purified using the EZNA Endo-Free maxi-prep kit. Quantified phage and single-stranded (ss) phagemid DNA lysates from Examples 1, 2 and 3 were used for transfection.

The gene transfer capacity of single-stranded minivector DNA was measured in comparison to double-stranded plasmid DNA. Gene transfer was assessed in both the HEK293T (EGFR−) and HeLa (EGFR+) cell lines as these were the representative cell lines for later EGF+ phage transfections. HEK293T cells were seeded in 24-well plates at 1×10⁵ cells/mL, while HeLa cells were seeded at 5×10⁴ cells/mL. The following day, 1 μg of double-stranded DNA (dsDNA) or 2 μg of single-stranded DNA (ssDNA) was transfected into each well after complexing with commercial cationic polymer transfection reagent, TurboFect. The empty vector, pM13ori2, and helper phage M13KO7 alone, were also transfected as negative controls. Efficiency of gene transfer was assessed via the Luciferase Assay System and reported as raw luminescence.

Optimization of transfection of single-stranded minivector DNA and phage particles. The capacity for gene transfer was assessed for both purified ssDNA and phage-encapsulated phage over time in the HeLa cell line. Cells were seeded at 5×10⁴ cells/mL in 24-well plates.

Phage particles were added to a final concentration of 5×10⁷ virions/mL: mini-(luc), mini-egf-(luc), full-(luc), and full-egf-(luc). Phages were added alone or complexed with TurboFect. For the purified phagemid DNA, pGL3-CMV and precursor plasmids, pM13ori2.cmvluc and pSW10, were also transfected as positive controls. For the phagemid particles, helper phage alone was also transfected as a negative control. Gene expression was assessed at 24, 48, 72, and 96 h via the Luciferase Assay System and reported as raw luminescence.

Assessment of cell viability after exposure to phage particles. HeLa cells were seeded in 96-well plates (Thermo Fisher Scientific) at 5×10⁴ cells/mL. The following day, EGF-displaying (M13SW7-EGF) and non-displaying (M13KO7) phage were transfected into each well at concentrations between 5×10⁵ and 5×10¹⁰ virions/mL. As a negative control, dimethyl sulfoxide (DMSO) was added at a concentration of 12%. Cell viability was assayed using 3-(4,5-dimethylthiazol-2-yl)-2,5-diphenyltetrazolium bromide (MTT) after 24 and 96 h. Conversion of MTT to formazan was allowed to proceed for 1 h at 37° C., at which point 100 μL of DMSO was added to dissolve the formazan. The absorbance of each well was taken at 492 nm on a Varioskan LUX multimode plate reader. Cell viability was reported as a percentage of the difference between the absorbances of each sample (Asample) and negative control (Anegative) relative to the absorbance of the untreated control (ANTC): A_{NTC}: (Aₛₐₘₚₗₑ−A_{negative})/A_{NTC}.

No significant difference was observed in cell viability for all transfections with purified DNA from untreated control. Turbofect did not impact cell viability and treatment with EGF and EGF phage did not impact cell viability. Cell viability was not adversely impacted by phage administration even at high concentrations of phage, regardless of EGF display.

Localization of transfected phage particles. Phage particles displaying EGF (M13SW7-EGF) or not displaying EGF (M13KO7) were purified and concentrated with polyethylene glycol (PEG) in phosphate-buffered saline (PBS), then labeled with Alexa Fluor 488. HeLa cells were seeded in 24-well plates at 5×10⁴ cells/mL. The following day, labeled phage particles and mini-(gfp) were transfected into cells at 5×10⁷ virions/mL. Wells were imaged 1, 6, 24, 48, 72, and 96 h after transfection. To image, cells were first fixed with 4% paraformaldehyde and permeabilized with 0.1% Triton-X. Nuclei were stained with 4′,6-diamidino-2-phenylindole (DAPI), while actin was stained with rhodamine phalloidin. Fixed cells were imaged on the EVOS FL Auto Imaging System at 40× magnification using the DAPI (nuclei), red fluorescent protein (RFP) (actin), and GFP (phage or expressed GFP) channels.

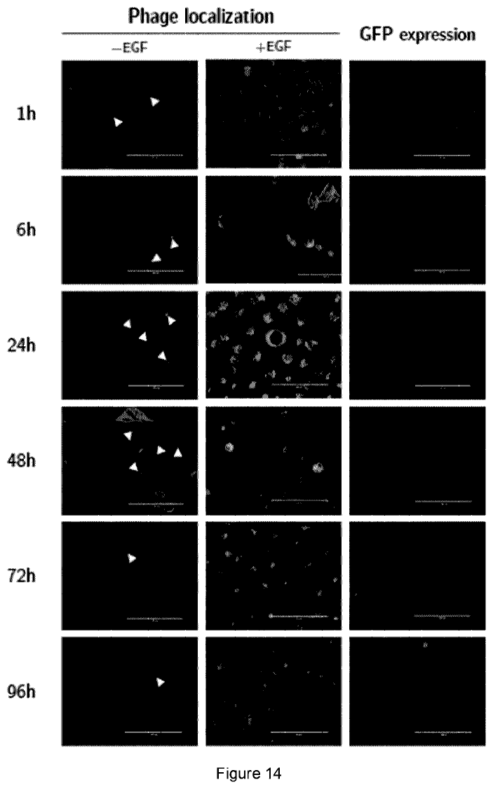

FIG. 14 shows Alexa Fluor 288-tagged M13SW7-EGF (EGF+) or M13KO7 (EGF−) in HeLa cells at 1, 6, 24, 48, 72, and 96 h post-treatment. The rightmost column also shows fluorescence from GFP expression over the same timeframe after administration of mini-egf-(gfp)miniphagemid. As can be seen, EGF-displaying phage were abundantly localized to the cell surface within an hour of administration in a ligand-dependent manner. In contrast, the same cannot be said for M13KO7. After 6 h, EGF-displaying phage can be seen surrounding the nucleus, indicating successful cell uptake and cytoplasmic translocation. Cellular localization of phages was observed within 1 h of administration. Within 1 h, phages were likely already internalized.

Alexa Fluor 488-tagged M13SW7-EGF were detectable even after 96 h, although it is unclear if the phage particles are still intact. Consistent with the results from the luciferase assays, expression of GFP from mini-egf-(gfp) is detectable at approximately 72 h post transfection. In contrast, few M13KO7 particles were detected even by 96 h. The cell-targeting ligand influences cellular localization and internalization.

The capacity of minivectors and full vectors for DNA transfer was compared across four cell lines known to express EGFR: HeLa, HT-29, MRC5, and A549. Cell lines were seeded at the following cell densities in 24-well plates: 5×10⁴ cells/mL (HeLa), 1×10⁵ cells/mL (HEK293T, MRC-5, HT-29). Phage particles were added to a final concentration of 5×10⁷ virions/mL. Helper phage alone (M13KO7) was transfected as a negative control. To assess the influence of a cationic lipid transfection carrier, phage particles were also complexed with 2 μL of TurboFect. Transfection was assessed by fluorescence microscopy on the EVOS FL Auto Imaging System or quantified through luminescence after 96 h. Luminescence was normalized against whole protein content, which was estimated via a bicinchoninic acid (BCA) assay. The efficiency of gene transfer was reported as luminescence per 100 μg of whole protein content.

Fold differences in luciferase activity from transfection of the minivectors and full vector phagemids generally show a positive increase in gene expression as a result of phagemid miniaturization into a minivector (Table 16). More concretely, fold differences in luminescence between EGF-displaying and non-displaying counterparts generally show a dramatic increase in gene expression from the display of EGF (Table 17).

Presence of the EGF receptor across the cell lines HEK293T, HeLa, HT-29, MRC-5, and A549 was visualized via Western blot.

### Transfection of Purified Single-Stranded Minivector DNA

To determine the optimal length of time for the assessment of single-stranded DNA transfection, purified plasmid dsDNA and minivector ssDNA were transfected over a period of 96 h in a HeLa cell line. The empty backbone vector pM13ori2 was used as a negative control, while pGL3-CMV (the source of the cmv-luc cassette) was used as a positive control. No background luminescence was observed after transfection with pM13ori2. Gene expression was also rapid: luciferase activity peaked approximately 24 to 48 h post-transfection. No significant difference was observed between any of the three vectors. For purified ssDNA, the onset of gene expression was delayed and not as potent. Luciferase activity peaked around 72 to 96 h; overall, gene expression as measured by luciferase activity was approximately 100-fold lower for purified minivector ssDNA compared to each plasmid counterparts. Notably, transfection of purified mini-(luc) ssDNA was correlated with increased luciferase activity over purified full-(luc) ssDNA.

Purified ssDNA from mini-(luc) and full-(luc) lysates were transfected into HEK293T as well as HeLa confirm ssDNA expression at 96 h in another cell line. No significant differences were observed between the plasmid vectors; furthermore, all plasmid vectors performed comparably well across both HEK293T and HeLa. Purified dsDNA transfects significantly better than ssDNA across both cell lines. Purified mini-(luc) ssDNA resulted in higher gene expression than purified full-(luc) ssDNA, resulting in a ten-fold increase in expression in HEK293T. ssDNA is a viable material for gene transfer, albeit at lower efficiency than ds plasmid DNA.

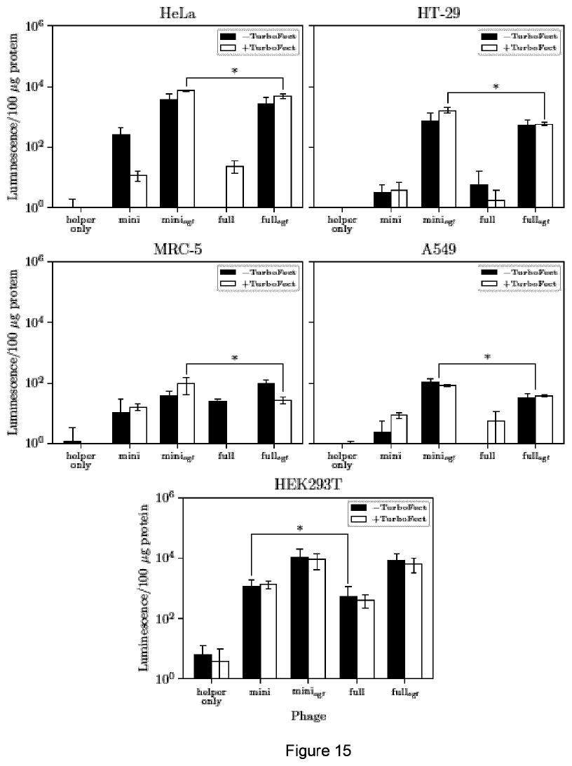

FIG. 15 shows luciferase gene expression of phage-delivered transgenes. Cell lines HeLa, HT-29, MRC-5, A549, and HEK293T were treated with minivectors encoding cmv-luc. The display of the cell-specific ligand EGF was compared to phagemids without any display. Gene expression is reported per 100 μg of whole protein content. The impact of the commercial cationic lipid reagent, TurboFect, was also assessed. Error bars represent SD, n=3. The * above the bars indicates a difference at significance level p<0:05.

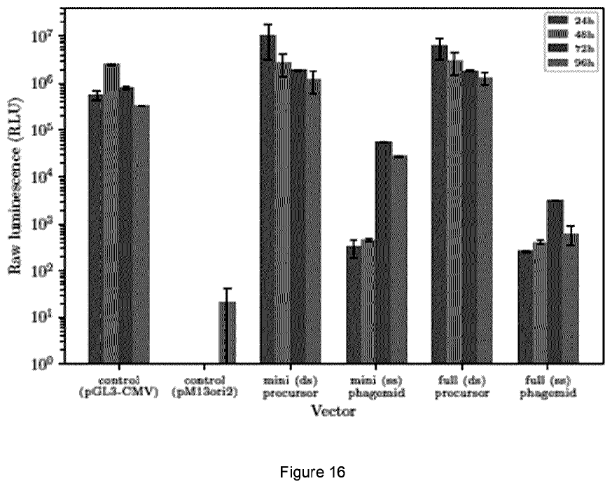

FIG. 16 shows gene expression over 96 h. HeLa cells were treated with 1 μg of ds purified plasmid DNA or 2 μg of ss purified phagemid DNA, respectively. DNA was complexed with TurboFect in all cases. Raw luminescence was reported 24, 48, 72, and 96 h after transfection. Error bars represent SD, n=3. Gene expression was rapid and peaked around 72 to 96 hours.

HeLa cells were treated with 5×10⁷ phage/mL. Raw luminescence was reported 24, 48, 72, and 96 h after transfection. Luciferase activity was assessed after treatment with purified miniphagemid particles in the HeLa cell line overtime. Gene expression from transfection of purified mini-(luc), mini-egf-(luc), full-(luc), and full-egf-(luc) was measured based on luciferase activity, alongside helper phage alone as a negative control. Peak luciferase activity was approximately ten-fold lower than the levels observed for the purified phage ssDNA. This is likely due to the differences in number of molecules transfected. Transfection of 2 μg of mini-(luc) ssDNA results in approximately 1×10⁹ phagemid DNA copies available per millilitre, roughly two orders of magnitude higher than the 5×10⁷ phage transfected per millilitre here. Due to practical constraints on phage volume, this concentration was selected to minimize the volumes of phage transfected per lysate. Quantification of lysates showed that full-(luc) and full-egf-(luc) lysates in particular were mostly comprised of helper phage (Example 2). In order to transfect equal amounts of cmv-luc encoding phage without transfecting exorbitant volumes of full phagemids, this amount was chosen as a compromise.

Negligible luciferase activity was observed from transfection of the full phagemid without EGF display; it was comparable to that of the negative control (helper only). In contrast, transfection of the mini-(luc) phagemid resulted in luciferase gene expression. For both phagemids, display of EGF increased gene expression. Without being bound by theory it is postulated that receptor-mediated cell internalization improves phage-mediated DNA transfer. Overall, increasing luciferase activity was observed until the end of the analysis period at 96 h.

Transfection efficiency of miniphagemids. Transfection was also performed in an EGFR− cell line, HEK293T. EGFR expression was verified via Western blot and relative levels of EGFR expression were reported using p-actin as the loading control. The EGFR+ cell lines appear to have very similar levels of EGFR; differences between the EGFR+ cell lines were not significant. EGFR expression in HEK293T was negligible, as expected.

In general, luciferase activity was highest in HEK293T and HeLa. The biggest factor contributing to increased gene expression was the display of the cell-specific ligand. The miniphagemid also conferred increased luciferase activity as compared to its full counterpart. This increase in gene expression was statistically significant in combination with the display of EGF and when complexed with TurboFect in cell lines HeLa, HT-29, and A549 (p<0.05).

TABLE 10
Fold difference in luciferase expression between
minivector and full vector phagemids

| | Fold difference in gene expression (mini/full) | | | |
|---|---|---|---|---|
| | EGF⁻ | | EGF⁺ | |
| Cell line | −TurboFect | +TurboFect | −TurboFect | +TurboFect |
| HEK293T | 5.22 ± 5.63 | 4.21 ± 3.79 | 2.03 ± 2.59 | 1.78 ± 1.79 |
| HeLa | ∞ | 0.51 ± 0.12 | 1.98 ± 1.92 | 1.48 ± 0.37 |
| HT-29 | ∞ | 7.44 ± 7.63 | 1.74 ± 1.40 | 2.81 ± 0.67 |
| MRC-5 | 0.52 ± 1.04 | ∞ | 0.43 ± 0.33 | 3.51 ± 1.73 |
| A549 | ∞ | ∞ | 3.20 ± 0.05 | 2.08 ± 0.18 |

TABLE 11
Fold difference in luciferase expression between
EGF-displaying and non-displaying phage

| | Fold difference in gene expression (EGF⁺/EGF⁻) | | | |
|---|---|---|---|---|
| | mini | | full | |
| Cell line | −TurboFect | +TurboFect | −TurboFect | +TurboFect |
| HEK293T | 11.73 ± | 7.17 ± | 43.94 ± | 17.65 ± |
| | 11.07 | 4.24 | 59.44 | 10.54 |
| HeLa | 25.59 ± | 704.94 ± | ∞ | 261.37 ± |
| | 33.01 | 351.36 | | 182.45 |
| HT-29 | ∞ | ∞ | ∞ | ∞ |
| MRC-5 | ∞ | 5.73 ± | 4.14 ± | ∞ |
| | | 2.79 | 0.47 | |
| A549 | ∞ | 9.96 ± | ∞ | ∞ |
| | | 3.34 | | |

In the case of the EGFR− control, HEK293T, mini-(luc) was correlated with increased luciferase activity over full-(luc) when complexed with TurboFect (p<0.05); while the difference in gene expression between mini-egf-(luc) and full-egf-(luc) trended similarly, it was not statistically significant. Display of a cell-targeting ligand was more impactful on resultant gene expression if the cell line expressed the targeted receptor. Gene expression was maximally 700-fold greater in HeLa, and over 100-fold greater in HT-29 when the minivector displayed EGF. The addition of TurboFect also resulted in positive enhancement of gene transfer. Minivectors complexed with TurboFect performed better than their full counterparts only if the minivector also displayed EGF. Specifically, mini-egf-(luc) complexed with TurboFect tended to perform better than full-egf-(luc) with TurboFect across all cell lines (p<0.05). However, in A549, the difference was significant regardless of the addition of TurboFect. On the whole, the minivector increased gene expression approximately between 2 to 3-fold.

Additionally, cells transfected by mini-egf-(gfp) or full-egf-(gfp) were visualized by GFP fluorescence 96 h after transfection. Cells transfected by EGF-phage were also examined. Few cells exhibited any fluorescence when transfected by phage without the targeting ligand. While some GFP fluorescence was detectable across all cell lines, it was generally weak. Although consistent with our observations of luciferase activity where gene expression from ssDNA (purified or phage-encapsulated), these results show that the efficiency of gene transfer per cell can be improved using minivectors.

Robust luciferase expression was observed in HEK293T, which does not express detectable levels of EGFR. In this cell line, although greater gene expression was observed with the EGF-displaying phage, the difference in gene transfer imparted by EGF+ versus EGF− phagemids was not statistically significant. However, regardless of EGF display, the miniphagemid was associated with greater luciferase activity: up to a 5-fold increase in luciferase activity over its full counterpart.

### Single-Stranded Minivector Improves Gene Transfer

Minivector phage particles were constructed which display EGF as a cell-specific ligand and encoding transgene cassettes that express the luciferase (cmv-luc) and green fluorescent protein (GFP), (cmv-gfp) reporters. Both purified and phage-encapsulated minivector DNA were correlated with greater luciferase activity over their full phagemid counterparts. Regardless of EGF display, the minivector was associated with greater luciferase activity: up to a 5-fold increase in luciferase activity over its full counterpart. Reduction in phagemid size (i.e. minivector) through removal of the bacterial backbone was found to improve phage-mediated gene transfer. Without being bound by theory it is possible that gene transfer was improved by increasing cytoplasmic diffusion towards the nucleus.

Internalization of EGF-displaying phage was observed to lead to juxtanuclear localization, likely via the lysoendosomal pathway. This was associated with improved gene transfer in EGFR+ cell lines, particularly in HeLa, over phage that did not display a ligand. Internalization of EGF-displaying phage was observed to lead to juxtanuclear localization, likely via the lysoendosomal pathway. This was associated with improved gene transfer in EGFR+ cell lines, particularly in HeLa, over phage that did not display a ligand. In addition, the display of a cell-specific targeting ligand enhanced gene transfer, likely as a result of ligand-receptor endocytosis.

### Example 5

### M13-Mediated Assembly of a Linear Covalently-Closed Double-Stranded DNA Minivector

A sense-antisense (SAS) minivector precursor was constructed that contains both the sense and antisense sequences of a transgene cassette approximately 2.0 kb in length, flanked by separated initiation and termination domains of the f1 functional origin (f1 ori). Insertion of a linker (spacer region) between the sense and antisense sequence helped stabilize the molecule during the cloning of the SAS construct. Helper phage M13KO7-mediated rescue of a double-stranded minivector was shown. The resulting lysate was subjected to HAP chromatography to isolate a LCC double-stranded minivector for downstream applications. The sense-antisense (SAS) precursor plasmid comprises a repeat of the cmv-gfp gene cassette inserted in reverse, such that both the sense and antisense sequences were present on one DNA strand. Rescue of the minivector from this SAS precursor demonstrated the assembly and production of a dsDNA filamentous phagemid minivector phage particle.

Construction of a Minivector Precursor with Both Sense and Antisense Transgene Cassettes Using Golden Gate Assembly

Two approaches were taken to construct the SAS minivector precursor encoding inverted repeats of the mammalian transgene cassette, cmv-gfp. The first was through Bsal-dependent Golden Gate Assembly and incorporated a small central region absent of self-complementarity (50 bp). The “sense” plasmid pM13ori2.cmvgfp was used both as the template and target destination vector. A Bsal recognition site within the ApR marker of the pM13ori2 backbone was previously mutagenized. Both target destination vector and the “antisense” cmv-gfp cassette were amplified from pM13ori2.cmvgfp with Bsal recognition sites in the primer overhangs. Digestion by Bsal and ligation by T4 ligase proceeded within the same reaction using the Golden Gate Assembly kit to generate the final construct pM13ori2.cmvgfp.SAS (GG). The ligation was transformed into Stbl4 and selected on LB agar supplemented with ampicillin.

Construction of a Minivector Precursor with Both Sense and Antisense Transgene Cassettes Using Traditional Endonuclease Digestion

A second approach used traditional restriction and ligation, and incorporated a larger central region absent of self-complementarity by way of the transit cloning vector pJET1.2 from the CloneJET PCR Cloning Kit. The cmv-gfp cassette, including the Pacl site, from pM13ori2.cmvgfp was first inserted into pJET1.2. The cassette was then excised from pJET1.2 using endonucleases BamHI and Hindlll, which included a 264 bp region of pJET1.2, referred to as the linker. Digestion of the destination vector pM13ori2.cmvgfp (containing the “sense” cassette) was performed using the same enzymes, both of which are located near the M13 STOP. The “antisense” fragment was then ligated using T4 ligase into the digested pM13ori2.cmvgfp to construct the SAS precursor with linker: pMl3ori2.cmvgfp.SAS (L). The ligation was transformed into Stbl4 and selected on LB agar supplemented with ampicillin. Additionally, the linker was later excised by digestion with Pacl to construct a precursor variant 20 without a linker: pM13ori2.cmvgfp.SAS (NL). Putative clones were subjected to endonuclease digestion to verify construction. Digested products were visualized via agarose gel electrophoresis (AGE).

Production and Purification of Phage and Episomal RF, Minivector dsDNA

A fresh E. coli colony carrying the sense-antisense precursor plasmid was inoculated into rich media and incubated at 37° C. with aeration until slightly turbid (0.01<A600<0.4). The culture was then infected with helper phage M13KO7 at a concentration of 1×10⁸ PFU/mL and returned to the same conditions for an additional 1-1.5 h. It was supplemented with kanamycin (70 μg/mL) and returned to the same conditions overnight. The next day, the culture was centrifuged (8000×g) to separate the bacterial pellet (replicative factor (RF) DNA) from the supernatant (phage lysate). After removal of the cell pellet, the phage lysate was purified through a 0.45 μm filter to remove residual bacterial debris.

As needed, phage were precipitated with polyethylene glycol (PEG) and re-suspended in Tris-NaCl (TN) buffer. Filtered lysate was concentrated through precipitation with PEG: 1/5 of the lysate volume was added in PEG, and the mixture was incubated at 4° C. for at least 2 h. The lysate was then centrifuged at 12 000×g for 15 min at 4° C. to separate the PEGylated phage (pellet) from media (supernatant). The pellet was then resuspended in a smaller volume of ice-cold TN buffer and a second PEG precipitation was carried out. PEG-precipitated phage suspensions were treated with DNase I to remove any extraneous phage or bacterial DNA in the sample. The concentrated phage lysate was stored at 4° C.

Episomal RF dsDNA were extracted from the pellet with the Monarch Plasmid Miniprep Kit. Minivector DNA were extracted from the phage lysate through phenol-chloroform extraction. Phenol was added to PEG-concentrated phage lysate (1:1, v/v) and mixed by vortexing. After centrifugation for 5 min at 4° C., the top aqueous layer was extracted. This layer was then extracted again with an equivalent volume of phenol:chloroform twice, and chloroform once. Finally, the minivector ssDNA was precipitated overnight with 100% ethanol at −80° C. The precipitated DNA was washed with 70% ethanol, dried, and re-suspended in RNase- and DNase-free water.

Extracted DNA was analyzed on a NanoDrop 2000 to determine concentration and purity. Extracted RFs were linearized through digestion with BamHI and visualized via AGE, while phagemid ssDNA was visualized directly via AGE without digestion._Mung bean nuclease preferentially and nonspecifically digests single-stranded DNA and was used to confirm the form of DNA in phage lysates arising from the SAS precursor vector. Purified DNA was subjected to mung bean nuclease (1 or 10 units) for 0.5 or 2 h (New England BioLabs). Digested samples were then visualized via AGE.

SAS phage production and RF processing. As a baseline, cultures were infected with helper phage M13KO7 at A600=0.02 to a final concentration of 1×10⁸ PFU/mL for 16 h before phage purification or RF extraction. Plasmids and RFs were extracted and purified from cell pellets using the Monarch Plasmid Miniprep Kit. RFx DNA was analyzed via AGE.

Separation of DNA species. The phage lysate arising from helper phage action on the SAS precursor vector contained both ssDNA helper phage genome and double-stranded LCC minivectors. Helper phage genome was separated from the dsDNA SAS minivector in the phage lysate using hydroxyapatite (HAP) chromatography, according to the method described by Fadrosh et al 2011 and Andrews-Pfannokoch et al., 2010. Briefly, the DNA mixture was incubated with 0.12 M phosphate buffer. The mixture was passed through hydrated HAP (Bio-Gel HTP hydroxyapatite) in an open glass Econo-column (Bio-Rad) at 60° C. DNA was eluted from the column in fractions of increasing phosphate concentration: 1) 0.12 M (ssDNA), 2) 0.2 M, and 3) 0.4 M and 1.0 M (dsDNA). Eluted fractions were extracted using phenol:chloroform (1:1, v/v), followed by chloroform (1:1, v/v), and desalted using Amicon Ultra Centrifugal Filter Devices (30,000 MWCO; Millipore, Billerica MA). Finally, nucleic acids were precipitated overnight with 100% ethanol at −80° C. The precipitated DNA was washed with 70% ethanol, dried, and re-suspended in RNase and DNase-free water.

Transfection of phage and purified DNA into HeLa. HeLa cells were seeded at 5×10⁴ cells/mL in a 24-well plate. Purified double-stranded plasmid DNA was complexed with TurboFect for 15 min prior to transfection and transfection was visualized by fluorescence microscopy.

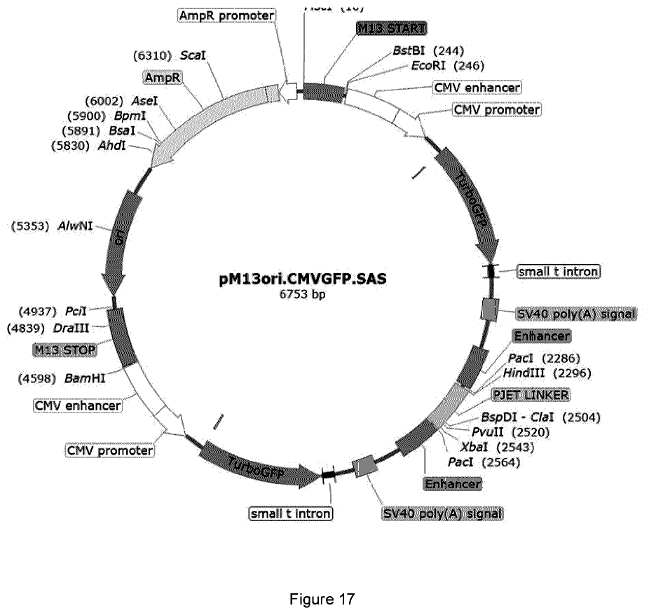

FIG. 17 shows an exemplary SAS minivector precursor construct (TurboGFP Reporter). A plasmid was constructed with a large interrupted DNA palindrome approximately 4 kb in size in Stbl4, an E. coli strain designed for the cloning of unstable inserts. The stable propagation of such a large perfect palindrome has not yet been reported to our knowledge. The addition of a linker between the sense and antisense sequences stabilized the large palindromic SAS clones while removal of the linker destabilized cloning of the SAS precursor vector. The Stbl4 host background in conjunction with a linker permitted the cloning and propagation of a large interrupted palindrome. Stable transformation and propagation of the final construct pM13ori2.cmvgfp.SAS (L) was accomplished in recA strain, JM109. Phage-mediated replication of this phagemid particularly is adversely affected in Stbl4.

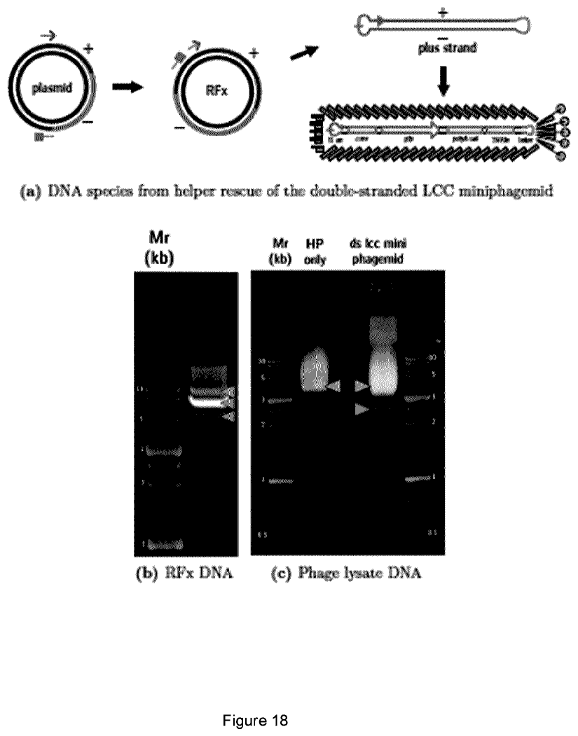

FIG. 18 shows the rescue of sense-antisense cassette by phage proteins. Cells were infected carrying the pM13ori2.cmvgfp.SAS (L) precursor vector with helper phage M13KO7. The schematic in FIG. 15(a) illustrates the species expected from helper phage rescue of a double-stranded LCC miniphagemid from the SAS precursor vector: 1) The split f1 on in the precursor vector (plasmid) is acted upon by phage proteins to form 2) a double-stranded RFx with both the sense-antisense sequences that undergoes rolling circle amplification to form 3) an ssDNA species whereby the sense and antisense regions can anneal to form a dsDNA molecule for assembly into 4) a double-stranded LCC minivector. Double-stranded RF products (b) and single-stranded purified phagemid DNA (c) were visualized via AGE. Bands are indicated as helper genome (top arrows in (b) and (c)), precursor plasmid (middle arrow in (b)), and RFx or ds-mini-(gfp) (lower arrows in (c)). The recombinant RF, which was predicted to be approximately 4.5 kb and the ds-mini-(gfp) minivector, which should be approximately 2.5 kb were present in the dsDNA RF and the ssDNA phage lysate.

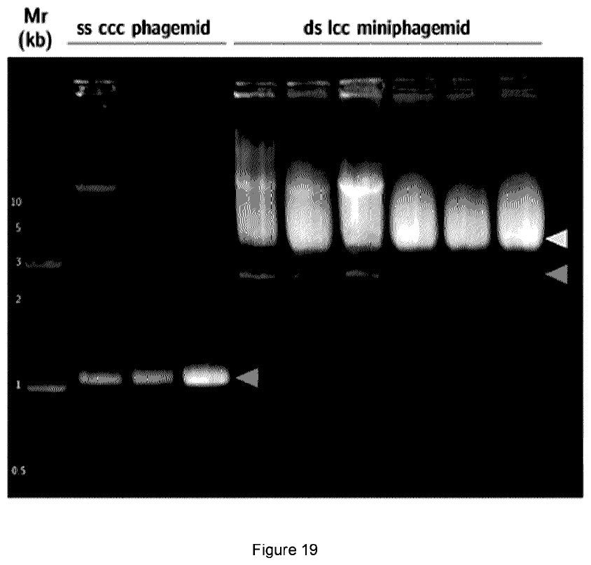

FIG. 19 shows M13KO7-mediated rescue and assembly of the CCC single-stranded cmv-gfp minivector (ss cccphagemid) and LCC dsDNA minivector (ds Icc miniphagemid). On the left, CCC purified single-stranded minivector was separated from helper phage genome via AGE. On the right, putative LCC dsDNA minivector was separated from helper phage genome. A faint band can be seen in each lysate that corresponds to the expected size of the double-stranded cmv-gfp minivector. Bands are indicated as helper genome (upper arrow on right) and ds-mini-(gfp) (lower arrow on right and single arrow on left). Helper phage M13KO7 packaged a double-stranded minivector. The AGE analysis shows that a majority of the ds-mini-(gfp) lysate is helper phage genome. M13KO7 preferentially assembles its own genome into phage progeny, which is expected as phage proteins exert optimal activity on ssDNA.

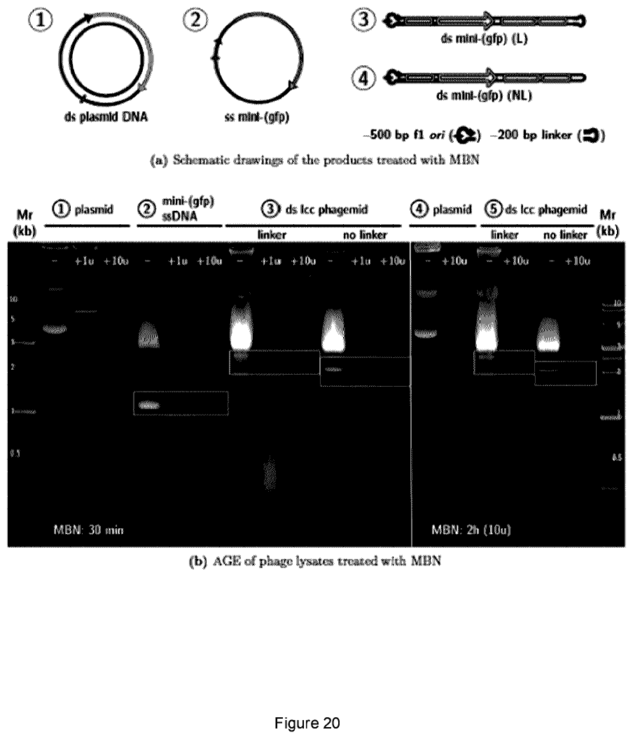

FIG. 20 shows AGE of phagemid and plasmid constructs following treatment with mung bean nuclease for 30 minutes and 2 hours. The SAS phage product is resistant to mung bean nuclease. Phagemid and plasmid constructs shown in the schematic of (A) were subjected to digestion with MBN for 30 min or 2 h and visualized via AGE (B). Lysates were treated for 30 min with 1 or 10 units of MBN: 1) CCC plasmid dsDNA, 2) CCC mini-(gfp) ssDNA minivector, 3) putative LCC ds-mini-(gfp) minivector (with or without a linker). The boxes enclose the bands expected to be phagemid DNA. Lysates were treated for 2 h with 10 units of MBN: 4) plasmid DNA, 5) ds-mini-(gfp) minivector (with or without a linker).

To verify if the 4.5 kb band in the phage lysate was double-stranded in nature, it was treated with mung bean nuclease (MBN), which preferentially cuts ssDNA. Like plasmid DNA, both ds-mini-(gfp) (L) and (NL) were resistant to MBN treatment. Even with 1 unit of MBN, all helper phage genome was degraded after 30 min, while the ds-mini-(gfp) remained almost completely intact. Importantly, prolonged treatment with MBN even degrades plasmid dsDNA, so it was expected that band intensity of the ds-mini-(gfp) would also decrease. With prolonged or increased MBN treatment, the band size corresponding to the dsDNA minivector decreased. This is suspected to be due to the MBN-mediated removal of ssDNA regions (the f1 on and the linker). Thus, we have demonstrated that the split origin of the sense-antisense precursor vector was a viable substrate for helper phage rescue of a double-stranded minivector.

The helper phage M13KO7 acts upon the split origin of the sense-antisense precursor plasmid to rescue a double-stranded minivector. Both the production of the RFx and the ds-mini-(gfp) were confirmed by AGE visualization of DNA bands at the predicted sizes. The duplex nature of the ds-mini-(gfp) was further confirmed by its resistance to MBN digestion. The decrease in size of the ds-mini-(gfp) after extensive MBN digestion is attributed to loss of single-stranded loop regions at the covalent ends of the molecule.

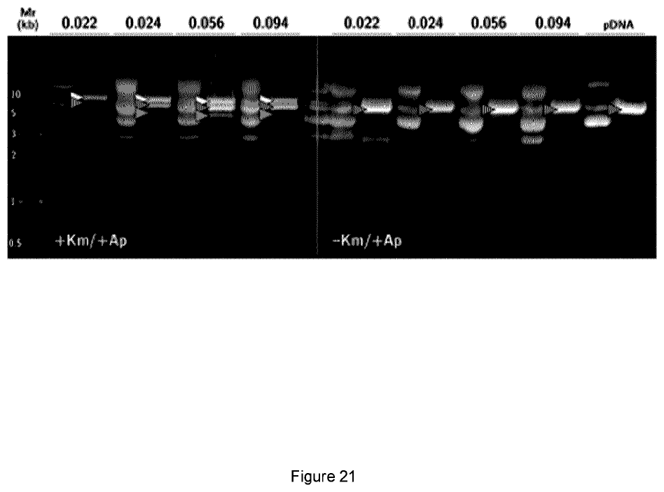

FIG. 21. Effect of optical density and helper antibiotic on SAS RFx conversion. Each sample was infected by M13KO7 at the optical density indicated above the wells. Purified RF DNA was run in pairs (undigested, BamHI-digested). Lysates on the left side were prepared from cultures supplemented both kanamycin and ampicillin. Lysates on the right side were not treated with kanamycin after addition of M13KO7. Arrows indicate bands of interest: top arrows on left side (helper genome), middle arrows on left and arrows on right (plasmid), lower arrows on left (RFx).

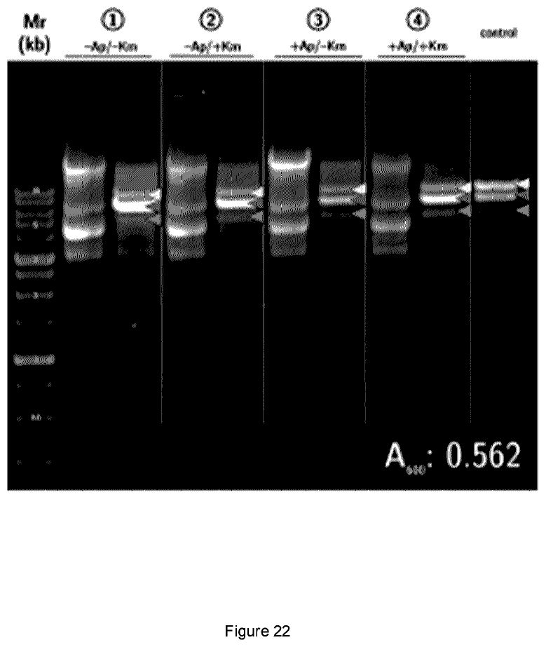

FIG. 22. Effect of phagemid and helper antibiotic on SAS RFx conversion. All samples were infected when A600=0.562. Samples were loaded in pairs (undigested, BamHI-digested). From left to right: 1) Ampicillin was removed from the media upon infection and kanamycin was not added; 2) Ampicillin was removed upon infection and kanamycin was added after infection; 3) Ampicillin remained in the media, but kanamycin was not added after infection; 4) Both antibiotics were present as in the original protocol. Arrows indicate bands of interest: top arrows (helper genome), middle arrows (plasmid), lower arrows (RFx).

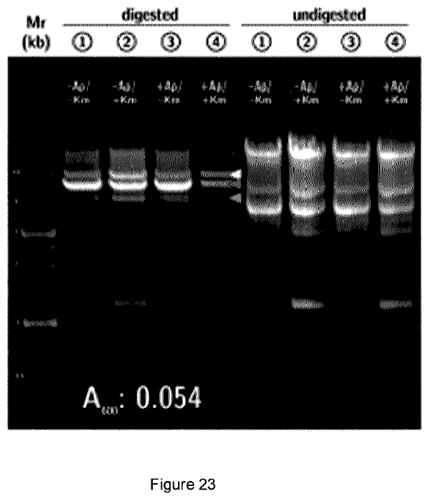

FIG. 23. Effect of antibiotic on SAS RFx conversion. All samples were infected when A600=0:054. Samples on the left were digested with BamHI while the same undigested samples are on the left. From left to right: 1) Ampicillin was removed from the media upon infection and kanamycin was not added; 2) Ampicillin was removed upon infection and kanamycin was added after infection; 3) Ampicillin remained in the media, but kanamycin was not added after infection; 4) Both antibiotics were present as in the original protocol. Arrows indicate bands of interest: upper arrow (helper genome), middle arrow (plasmid), lower arrow (RFx). Ampicillin and kanamycin are dispensable when cells were in late log phase upon infection.

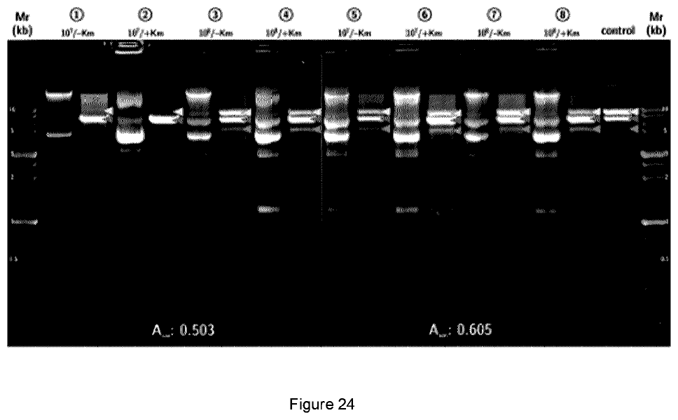

FIG. 24. Effect of antibiotic, optical density, and MOI on SAS RFx conversion. On the left, samples were infected when A600=0.503; on the right, when A600=0.605. Samples were loaded in pairs (undigested, BamHI-digested). When the culture reached A600=0:503, helper phage M13KO7 was added at a concentration of 1×10⁷ PFU/mL without kanamycin (1) and with (2); and at a concentration of 1×10⁸ PFU/mL without kanamycin (3) and with (4). When the culture reached A600=0:605, helper phage M13KO7 was added at a concentration of 1×10⁷ PFU=mL without kanamycin (5) and with (6); and at a concentration of 1×10⁸ PFU/mL without kanamycin (7) and with (8). Arrows indicate bands of interest: top arrows in each lane (helper genome), middle arrows in lanes with three arrows and bottom arrows in lanes with two arrows (plasmid), bottom arrows in lanes with three arrows (RFx). Cultures infected with M13KO7 at A600=0.505 or 0.605 to a final concentration of 1×10⁷ or 1×10⁸ PFU/mL. Production of the RFx appeared robust even when at A600=0.605, when cells may begin to enter stationary phase. The increase in helper phage concentration appeared to be correlated with improved RFx production, particularly in the presence of kanamycin.

Helper phage rescue of a sense-antisense RFx was demonstrated in JM109. The efficiency of filamentous production of dsDNA progeny particles in JM109 was improved by modifying the duration of infection. The optimal duration for infection was 16-18 h, as appreciable amounts of RFx or ssDNA via AGE were not observed when infection was stopped before 16 h or when it proceeded beyond 18 h. RFx was detected in most lysates if infection was allowed to proceed for 16-18 h, but were not observed outside this range.

Purified RF DNA was harvested from cultures carrying the SAS precursor plasmid infected by helper phage at optical densities from A600=0.022 to 0.094 (FIG. 19), A600=0.562 (FIG. 20), A600=0.054 (FIG. 21), A600=0.503 and A600=0.605 (FIG. 22).

Antibiotic selective pressure against the helper phage improved precursor-RFx conversion, while antibiotic selective pressure against the precursor vector after infection did not provide any improvements to the process. RFx production with or without antibiotic selective pressure (kanamycin) was examined for the helper phage. Notably, multiple bands of different sizes were detected; for example, a band slightly larger than 1 kb was seen both in the uncut and cut lanes; these bands are not present in the absence of helper phage. This may be indicative of recombination events stemming from replication from f1 ori, not the plasmid origin of replication (ori). Cultures were infected with M13KO7 at A600=0.054 with a combination of helper or precursor plasmid antibiotic selection. Although helper phage genome was present even in the absence of kanamycin, bands correlating to linearized RFx were distinctly fainter in these samples; thus, maintenance of helper phage with antibiotic selection appears to improve processing of the sense-antisense precursor. On the other hand, removal of ampicillin from the media did not appear to negatively impact RFx production; in fact, the brightest RFx band here was from a culture supplemented with kanamycin but devoid of ampicillin. The presence of kanamycin was most strongly correlated with good RFx production. A strong helper phage presence appears to overcome inefficiencies of processing the split origin around a DNA palindrome.

The brightest RFx band was associated with infection of culture at A600=0.056, when supplemented with kanamycin; no RFx production nor the presence of the helper phage in absence of kanamycin was detected at these optical densities. This suggests that antibiotic selection may help maintain helper phage activity. Strong bands correlating to the M13KO7 genome were seen in the absence of kanamycin, but only when cultures were infected at A600=0.562. Both ampicillin and kanamycin appeared dispensable when cells were in late log phase upon infection. Superior RFx production was observed at A600=0.05, 0.5, and 0.6 when helper phage added at 1×10⁸ PFU/mL in the presence of kanamycin. This corresponds to MOIs of 430, 60, and 111, respectively; it does not appear that MOI is a major impact on RFx production, although the absolute number of helper phage molecules may contribute.

### Hydroxyapatite Chromatography Separation of DNA Species

Separation of ds-mini-gfp vectors (LCC dsDNA minivectors) from genomic ssDNA and helper phage contamination in the phage lysate for downstream applications was done using hydroxyapatite chromatography. SAS minivector lysates were combined into 2 volumes for separation via HAP chromatography. Fractions were eluted from the four fractions as above, and visualized via AGE. The A260/280 ratios ranged from 1.80 to 1.97. The A260/230 ratios ranged from 1.56 to 2.49; the low end is indicative of the presence of contaminants that absorb strongly at 230 nm. The majority of ssDNA M13KO7 genome was recovered in the 0.12 M phosphate fraction, while the ds-mini-(gfp) minivector was recovered in the 0.4 and 1.0 M fractions.

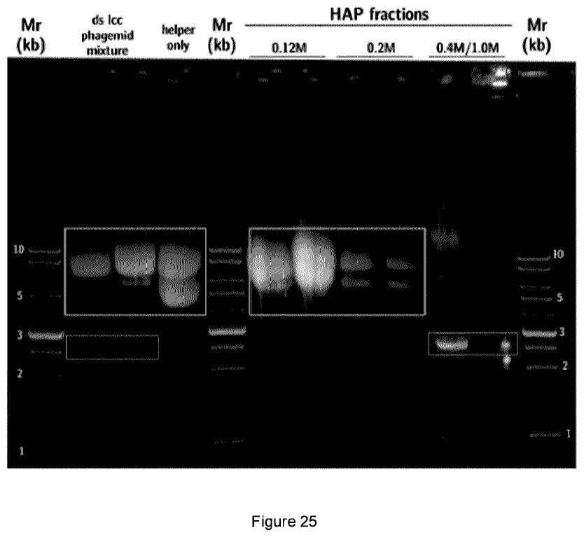

FIG. 25. Separation of the LCC dsDNA miniphagemid from ssDNA M13KO7 genome using hydroxyapatite chromatography. A mixture of helper genome and LCC dsDNA miniphagemid was subjected to HAP chromatography. The majority of ssDNA M13KO7 genome was recovered in the 0:12M phosphate fraction, while the ds-mini-(gfp) miniphagemid was recovered in the 0:4 and 1:0M fractions. Due to the overwhelmingly large amounts of ssDNA, some of it was also present in the 0:2M fraction. The upper boxes indicate helper phage ssDNA while the lower, smaller boxes indicate ds-mini-(gfp).

In complex media such as LB, E. coli are thought to be in steady-state growth, commonly referred to as the log phase, until approximately A600=0.6-1.0. It has even been reported that steady-state growth can stop much earlier, around A600=0.3, after which the growth rate and cell mass gradually decrease. Therefore, it was expected that infection by helper phage at low optical density should be superior; pili, the adsorption sites for M13, are lost as cells enter stationary phase. The start of stationary phase, as indicated by the rise of intracellular levels of stationary phase sigma factor (σS), begins around A600=0.5. However, robust RFx conversion was observed even when cells were infected between A600=0.5 and 0.6, which is indicative of sufficient pilus formation to support M13 infection even at this stage of growth.

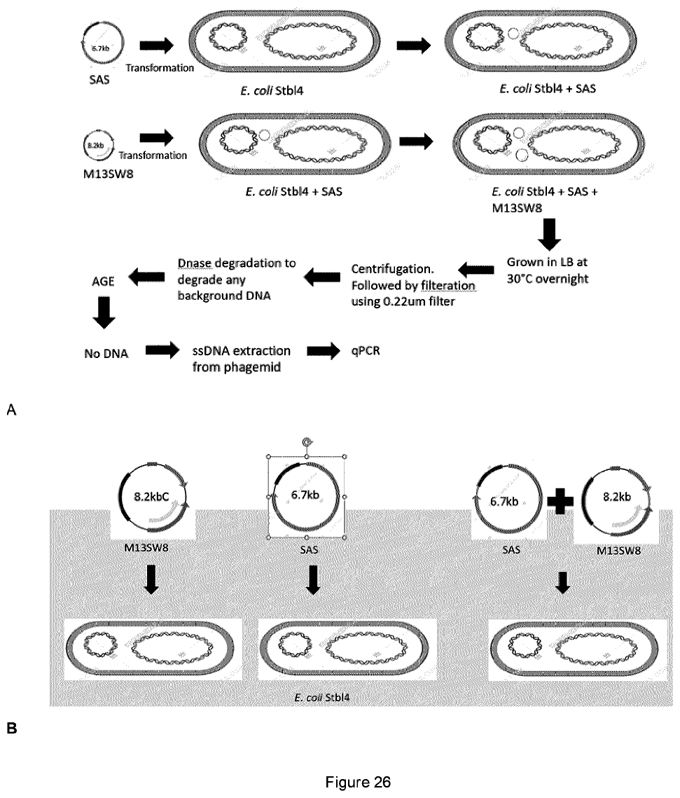

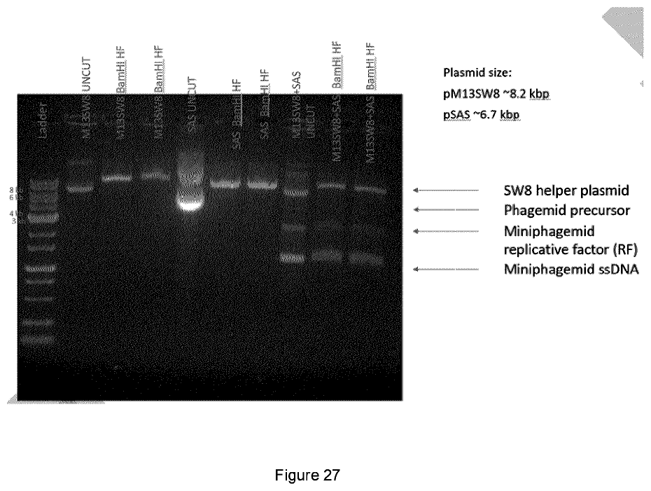

FIG. 26 shows a process of generating SenseAntiSense SAS miniphagemids using pM13ori.cmvgfp.sas, Stbl4 and the M13SW8 helper bacteriophage and the removal of contaminating helper DNA for analysis (A) and SAS phagemid production (cotransformation), (B). Generation of the SAS phagemid was confirmed by qPCR analysis (not shown) and agarose gel electrophoresis (FIG. 27). SenseAntiSense miniphagemids were produced without contamination of helper plasmid/phage in Stbl4 cells. SenseAntiSense miniphagemids are equally targetable as Sense miniphagemids which are also made without contamination of helper phage/plasmid using the M13SW8 helper plasmid.

### REFERENCES

Andrews-Pfannkoch, C.; Fadrosh, D. W.; Thorpe, J.; Williamson, S. J. Hydroxyapatite-mediated separation of double-stranded DNA, single-stranded DNA, and RNA genomes from natural viral assemblages. Applied and Environmental Microbiology 2010, 76, 5039-5045.
Beck and Zinck, 1981. Nucleotide sequence and genome organization of filamentous bacteiropahges in f1 and fd. Gene, 16, 35-58.
Dotto, G. P., Horiuchi, K, Zinder, N. D. 1984. The functional origin of bacteriophage f1 DNA replication. Its signals and domains. J. Mol. Biol. DOI: 10.1016/s002-2836 (84) 80020-0.
Fadrosh, D. W.; Andrews-Pfannkoch, C.; Williamson, S. J. Separation of Single-stranded DNA, Double-stranded DNA and RNA from an Environmental Viral Community Using Hydroxyapatite Chromatography. Journal of Visualized Experiments 2011, DOI:10.3791/3146.
Horiuchi, K. 1997. Initiation mechanisms in replication of filamentous phage DNA. Genes to Cells, 2, 425-432.
Larocca et al., 2001. Receptor-targeted gene delivery using multivalent phagemid particles. Molecular Therapy, 3, 476-484). Short, J. M., Fernandez, J. M., Sorge, J. A., Huse, W. D., 2003, Lambda ZAP: a bacteriophage lambda expression vector with in vivo excision properties. Nucleic Acids Research, 16: 7583-7600.
Short, J. M., Sorge, J. A. 1995. In Recombinant DNA methodology II Wu, R., Ed, Selected Methods in Enzymology. Academic Press. Boston, 185-198.

What is claimed is:

1. A minivector for use in delivering a deoxyribonucleic acid to at least one target cell, the minivector comprising the deoxyribonucleic acid and a filamentous phage origin of replication, wherein the minivector is otherwise devoid of prokaryotic vector backbone genetic sequences.

2. (canceled)

3. (canceled)

4. (canceled)

5. The minivector of claim 1, wherein the minivector is a linear covalently closed double stranded DNA minivector.

6. The minivector of claim 1, wherein said at least one target cell comprises a mammalian cell.

7. (canceled)

8. The minivector of claim 1, wherein the filamentous phage origin of replication is an M13 origin of replication or an f1 origin of replication.

9. (canceled)

10. (canceled)

11. A filamentous bacteriophage particle comprising the minivector according to claim 1, wherein the filamentous bacteriophage particle comprises a targeting moiety on the bacteriophage particle surface for targeting the bacteriophage particle to the at least one target cell.

12. (canceled)

13. A method of producing a minivector encapsulated in a filamentous bacteriophage, the method comprising:
infecting a bacterial host cell comprising a minivector precursor with a helper phage, wherein the bacterial host cell is capable of propagating filamentous bacteriophage and wherein the minivector precursor comprises a deoxyribonucleic acid flanked by initiation and termination elements of a filamentous bacteriophage origin of replication and wherein the helper phage is a filamentous bacteriophage;
culturing the bacterial host cell under conditions that promote replication and amplification of the minivector from the minivector precursor, packaging of the minivector into progeny bacteriophage and extrusion of the progeny bacteriophage from the bacterial host cell; and
harvesting extruded progeny filamentous bacteriophage encapsulating the minivector.

14. The method of claim 13, wherein the helper bacteriophage is f1, fd, M13, or M13KO7.

15. (canceled)

16. (canceled)

17. The method of claim 13, wherein the helper bacteriophage is derived from M13KO7 and has been modified to replace the packaging signal downstream of gIV and upstream of Tn903 with a Rho-independent terminator.

18. The method of claim 13, wherein the helper bacteriophage is M13SW8 and comprises the sequence set forth in SEQ ID NO:4.

19. (canceled)

20. (canceled)

21. The method of claim 13, wherein the minivector precursor comprises a polynucleotide comprising X-Y-Z,
wherein X comprises an initiation element of a filamentous phage origin of replication;
Y comprises a cassette comprising a deoxyribonucleic acid; and
Z comprises a termination element of the filamentous phage origin of replication.

22. (canceled)

23. (canceled)

24. The method of claim 21, wherein the deoxyribonucleic acid comprises a sense copy and an antisense copy of a target deoxyribonucleic acid, and the sense copy and the antisense copy of the target deoxyribonucleic acid are separated by a linker for stabilizing the minivector precursor during cloning.

25. (canceled)

26. (canceled)

27. (canceled)

28. (canceled)

29. (canceled)

30. (canceled)

31. The method of claim 13, wherein the bacterial host cell is an E. coli host cell comprising mutations in recA, endA, and/or gyrA genes.

32. (canceled)

33. The method of claim 13, wherein the bacterial host cell is an E. coli host cell, and the E. coli host cell is E. coli K12, JM109, or Stbl4.

34. (canceled)

35. A method of producing a minivector, the method comprising the method of claim 13, further comprising:
separating bacterial debris from harvested extruded progeny bacteriophage by centrifugation to produce a supernatant containing the bacteriophage lysate;
optionally purifying the supernatant containing the bacteriophage lysate by filtration to obtain a filtered lysate;
optionally concentrating the filtered bacteriophage lysate;
and
isolating the minivector from the filtered bacteriophage lysate.

36. A minivector produced by the method of claim 13.

37. (canceled)

38. (canceled)

39. (canceled)

40. (canceled)

41. A method of treating a disease or condition in a subject in need thereof, the method comprising:
administering the filamentous bacteriophage particle of claim 11 to the subject.

42. (canceled)

43. (canceled)

44. A minivector precursor for use in filamentous bacteriophage-mediated deoxyribonucleic acid delivery to a target cell, the minivector precursor comprising a polynucleotide comprising X-Y-Z, wherein X comprises an initiation element of a filamentous phage origin of replication;
Y comprises a cassette comprising the deoxyribonucleic acid for delivery; and
Z comprises a termination element of the filamentous phage origin of replication.

45. (canceled)

46. (canceled)

47. (canceled)

48. The minivector of claim 44, wherein the filamentous phage origin of replication is an M13 origin of replication.

49. The minivector of claim 44, wherein the filamentous phage origin of replication is an f1 origin of replication.


SEQUENCE LISTING (4 sequences)

SEQ ID NO: 1 - DNA, 2461 bp, synthetic construct
  source 1..2461: mol_type = other DNA
ccaatgatggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaatttaaagatatctcgagcttcgaattctgcagtctagattaattaagtcgacaagcttgacgtcggtaccgagctcgcccgggatccgtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaaatcggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgcatgccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 2 - DNA, 4480 bp, synthetic construct
  source 1..4480: mol_type = other DNA
ccaatgatggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaatttaaagatatctcgagcttcgaattccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccctcgagtcatgaactagtgcggccgcctgcaggtgaccagatctgatatcactcgtacgcgtctcggccggccaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaaagagtttaaacggtaaatataaaatttttaagtgtataatgtgttaaactactgattctaattgtttgtgtattttagatggccggctgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttacgatcgcccttcccaacagttgcgcaatcccgcccctaactccgcccagttccgcccattctccgccccatggctgactaattttttttatttatgcagaggccgaggccgcctcggcctctgagctattccagaagtagtgaggaggcttttttggaggccggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccttaattaagtcgacaagcttgacgtcggtaccgagctcgcccgggatccgtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaaatcggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 3 - DNA, 5054 bp, synthetic construct
  source 1..5054: mol_type = other DNA
ccaatgatggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaatttaaagatatctcgagcttcgaattcccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatctgcagggtaccgagctcgcccgggatccgtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaaatcggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgcatgccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 4 - DNA, 8224 bp, unidentified
  source 1..8224: mol_type = other DNA
aacgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgagctacagcaccagattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagcctttttttttggagattttcaacgtgaaaaaattattattcgcaattcctttagttgttcctttctattctcactccgctgaaactgttgaaagttgtttagcaaaaccccatacagaaaattcatttactaacgtctggaaagacgacaaaactttagatcgttacgctaactatgagggttgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgctaataattttgatatggttggttcaattccttccataattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgcacctaaagatattttagataaccttcctcaattcctttctactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaagattctggcgtaccgttcctgtctaaaatccctttaatcggcctcctgtttagctcccgctctgattccaacgaggaaagcacgttatacgtgctcgtcaaagcaaccatagtacgcgccctgtagcgagccataatggctaatcccacaggccgccagttccgctggcggcattttgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccgattcgagctcgccccggggatcgaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccctgcaggtctcgggctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagacctctcaaaaatagctaccctctccggcatgaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgtt